(19) United States
(12) Patent Application Publication
(10) Pub. No.: US 2025/0213658 A1
(43) Pub. Date: Jul. 3, 2025

(54) METHODS AND FORMULATIONS FOR INTRANASAL DELIVERY OF INSULIN IN THE TREATMENT OF DIABETIC EYE DISEASE

(71) Applicant: WAKE FOREST UNIVERSITY HEALTH SCIENCES, Winston-Salem, NC (US)

(72) Inventors: Sally Shin Yee Ong, Greensboro, NC (US); Rebecca Marie Sappington, Winston-Salem, NC (US)

(73) Assignee: WAKE FOREST UNIVERSITY HEALTH SCIENCES, Winston-Salem, NC (US)

(21) Appl. No.: 18/853,385

(22) PCT Filed: Jun. 1, 2023
(86) PCT No.: PCT/US2023/024209

§ 371 Date: Oct. 1, 2024

Related U.S. Application Data
(60) Provisional application No. 63/362,390, filed on Apr. 1, 2022.

(51) Int. Cl. A61K 38/28; A61K 9/00; A61P 27/02

(52) U.S. Cl. CPC A61K 38/28; A61K 9/0043; A61P 27/02

(57) ABSTRACT

In various aspects, methods and formulations are provided for treating subjects, in particular mammals, intranasally with an effective amount of insulin, an insulin analog, or an insulin derivative to treat or prevent an eye disease, in particular a diabetic eye disease such as diabetic retinopathy.

### CROSS-REFERENCE TO RELATED APPLICATIONS

This application claims priority to, and the benefit of, U.S. provisional application entitled “METHODS OF USING INSULIN FOR PREVENTING AND TREATING EYE DISEASE” having Ser. No. 63/362,390 filed Apr. 1, 2022, the contents of which are incorporated by reference herein.

### TECHNICAL FIELD

The present disclosure generally relates to the treatment of eye disease in patients in need thereof.

### BACKGROUND

Diabetic retinopathy (DR) is the most common microvascular complication in diabetes mellitus (DM) and the most frequent cause of acquired blindness in working age adults worldwide.¹⁵ The prevalence of DM is predicted to increase exponentially in the United States. In 2018, an estimated 34.1 million or 13.0% of American adults had DM.¹⁶ By 2050, DM is projected to affect 48.3 million American adults.¹⁷ As the prevalence of DM increases, the public health burden of DR also worsens. A pooled study in 2012 reported that 35% of Americans with DM had some form of DR, including 7% who had proliferative diabetic retinopathy and 7% who had diabetic macular edema.²⁰ Additionally, ten percent had vision-threatening stages of disease.²⁰ In 2016-2017. 8.6% of adults aged 45 or older with diagnosed diabetes had DR, while 4.1% had vision loss due to DR.¹ DR prevalence and complications are also higher in racial/ethnic minorities, including African Americans, Hispanics and American Indians.²¹

Diabetic Retinopathy (DR) can be classified into two broad categories: the earlier stages of nonproliferative diabetic retinopathy (NPDR) and the more severe stages of proliferative diabetic retinopathy (PDR). NPDR is diagnosed using clinical findings of microaneurysms, intraretinal hemorrhages, intraretinal microvascular abnormalities (IRMA) and venous caliber changes, and on its own, rarely affects vision. PDR is characterized by pathologic preretinal neovascularization which can be complicated by extensive preretinal or vitreous hemorrhage, or tractional retinal detachment, all of which can cause severe vision loss. An additional important classification is diabetic macular edema (DME), which can occur across all stages of NPDR and PDR, and is the most common cause of vision loss in DR. DME occurs as a consequence of blood retinal barrier breakdown, with resultant vascular leakage of fluid and circulating fluid into the neural retina.

Current treatment paradigms include intravitreal anti-vascular endothelial growth factor (VEGF) injections and laser which target the late stages of the pathway in DR pathogenesis (blockage of angiogenic and inflammatory factors), but these do not address the underlying pathobioloic cause of DR.² Up to 50% of patients with DME do not adequately respond to anti-VEGF therapy. Degeneration of retinal neurons (photoreceptors, bipolar cells, horizontal cells, amacrine cells and ganglion cells) and glia (astrocytes, Muller cells and microglial cells) has been implicated in DR.³⁻⁷ Importantly, insulin has been found to rescue these neurons and glia from apoptotic cell death.⁸⁻¹⁰

There remains a need for improved formulations and methods for the treatment of DR.

### SUMMARY OF THE DISCLOSURE

In accordance with the purpose(s) of the disclosure, as embodied and broadly described herein, the disclosure, in one aspect, relates to methods of treating or preventing one or more symptoms associated with eye disease, in particular diabetic retinopathy, in a mammal in need thereof via intranasal delivery of insulin or an insulin analog or derivative.

Further disclosed are a variety of pharmaceutical formulations, kits, and dosage forms for carrying out the methods of intranasal delivery of an effective amount of insulin (or an insulin analog or insulin derivative) to a mammal in need thereof to of treat or prevent one or more symptoms associated with eye disease, in particular diabetic retinopathy.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects may be usable in all aspects of the disclosure taught herein. Furthermore, the individual features of the dependent claims, as well as all optional and preferred features and aspects of the dependent claims are combinable and interchangeable with one another.

### BRIEF DESCRIPTION OF THE DRAWINGS

Further aspects of the present disclosure will be readily appreciated upon review of the detailed description, described below, when taken in conjunction with the accompanying drawings.

FIG. 1 depicts the distribution of FITC-insulin in ocular tissue sixty minutes after intranasal administration using fluorescent signal. FITC patterning, shown by stars, indicate deposition of insulin predominantly in the near outer segments of rods and cones. Lower intensity deposition of FITC-insulin depositions, shown by stars, were observed in the inner and outer plexiform layers and the nerve fiber layer.

FIG. 2 depicts the distribution of FITC-insulin in ocular tissue sixty minutes after intranasal administration using fluorescent signal. FITC patterning, shown by stars, indicate deposition of insulin predominantly in the retinal pigment epithelium. Lower intensity deposition of FITC-insulin depositions, shown by stars, were observed in the choriocapillaris.

FIG. 3 is a microscope image of retinal tissue from female Sprague Dawley rats that were fed ad-libitum sacrificed by trans-cardial perfusion one hour after FITC insulin administration. FITC insulin uptake (stars) was most pronounced in the retinal pigment epithelium and outer segments of rods and cones, followed by the inner plexiform and nerve fiber layers.

FIGS. 4A-4C are graphs of electroretinograms of 13-week old C57/BL6 and diabetic C57BL/KsJ-db/db mice taken before and at the end of intranasal saline or insulin treatment daily for 10 weeks. FIG. 4A is an electroretinogram of the negative control group consisting of C57/BL6 mice (n=4) group treated with intranasal saline. FIG. 4B is an electroretinogram of the positive control group consisting of C57BL/KsJ-db/db mice (n=3) treated with intranasal saline. FIG. 4C is an electroretinogram of C57BL/KsJ-db/db mice (n=4) treated daily with 2U of intranasal insulin.

FIG. 5 is a bar graph of the blood glucose level (mg/dl) for C57B6 mice and BKS.Cg-Dock7^{m}+/+Lepr^{db}/J diabetic mice before and after administration of either intranasal saline (positive and negative control groups), 1U Insulin (low dose group), or 2U Insulin (high dose group).

FIGS. 6A-6D are electroretinograms at a stimulus of 0.01 cd·s/m² for C57B6 mice (FIG. 6A) and BKS.Cg-Dock7^{m}+/+Lepr^{db}/J diabetic mice (FIGS. 6B-6D) before and after administration of intranasal saline (FIGS. 6A-6B), 1U Insulin (FIG. 6C), or 2U Insulin (FIG. 6D).

FIGS. 7A-7D are electroretinograms at a stimulus of 0.1 cd·s/m² for C57B6 mice (FIG. 7A) and BKS.Cg-Dock7^{m}+/+Lepr^{db}/J diabetic mice (FIGS. 7B-7D) before and after administration of intranasal saline (FIGS. 7A-7B), 1U Insulin (FIG. 7C), or 2U Insulin (FIG. 7D).

FIGS. 8A-8D are electroretinograms at a stimulus of 1 cd·s/m² for C57B6 mice (FIG. 8A) and BKS.Cg-Dock7^{m}+/+Lepr^{db}/J diabetic mice (FIGS. 8B-8D) before and after administration of intranasal saline (FIGS. 8A-8B), 1U Insulin (FIG. 8C), or 2U Insulin (FIG. 8D).

FIG. 9 is bar graphs of the b waves from the electroretinograms at a stimulus of 0.01 cd·s/m² (left panel), 0.1 cd·s/m² (middle panel), and 1 cd·s/m² (right panel) for C57B6 mice and BKS.Cg-Dock7^{m}+/+Lepr^{db}/J diabetic mice before and after administration of intranasal saline, 1U Insulin, or 2U Insulin.

FIG. 10 is bar graphs of the a waves from the electroretinograms at a stimulus of 0.01 cd·s/m² (left panel), 0.1 cd·s/m² (middle panel), and 1 cd·s/m² (right panel) for C57B6 mice and BKS.Cg-Dock7^{m}+/+Lepr^{db}/J diabetic mice before and after administration of intranasal saline, 1U Insulin, or 2U Insulin.

FIG. 11 is images of the toludine blue staining of the retina of for C57B6 mice and BKS.Cg-Dock7^{m}+/+Lepr^{db}/J diabetic mice after administration of intranasal saline, 1U Insulin, or 2U Insulin.

### DETAILED DESCRIPTION

The molecular and cellular pathways involved in DR pathophysiology are complex and remain under active investigation as researchers explore various targets for drug development. However, there is growing consensus that DR is a disease of the neurovascular unit, which refers to the interdependency of glia, neurons and vasculature to maintain normal retina function.^{22,23} Traditional research in DR pathogenesis has focused on breakdown of the vasculature leading to nonperfusion, hypoxia, loss of the blood retinal barrier and consequently DME and/or PDR. Death of endothelial cells, pericytes and smooth muscle cells causing progressive microvascular damage has been well documented in early DR.^{24,25}

In more recent years, however, there is increasing recognition of the important roles glia and neurons play in DR pathogenesis. Retinal blood vessels are made of endothelial cells, pericytes (capillary level), vascular smooth muscle cells (artery/arteriole level) and closely associated glia and neurons.²² In fact, neurodegeneration (apoptotic death of neurons and reactive gliosis) has been observed in the retina of diabetic donors in the absence of any vasculature changes.²² These findings suggest that neurodegeneration likely predates the microvasculature changes seen in DR.²² Moreover, observational reports have shown that neurodegeneration measured by multifocal electroretinogram can predict which locations would develop DR in the future.²⁶⁻²⁸

Reiter et al. had previously demonstrated that the retina has a high basal activity of the insulin receptor→phosphoinositide 3 kinase→Akt→p70^{S6} kinase pathway,²⁹ but this pathway becomes disrupted in insulin dependent diabetes with concomitant accelerated apoptosis of retinal neurons.⁸ Systemic, intravitreal and subconjunctival administration of insulin has been shown to restore prosurvival insulin receptor and Akt kinase activity, and decrease apoptotic cell death associated with diabetes.^{9,11,12} Meanwhile, insulin resistance and poor glycemic control have been found to be strongly linked to the development of DR, while rigorous insulin therapy has been associated with a decline in DR onset and progression.^{30,31}

There are numerous challenges associated with current routes of insulin delivery. Insulin has a short half-life in plasma,³² and it is difficult to administer enough systemic insulin to reduce the risk of retinopathy without causing hypoglycemia.³³ Topical eye drops are not effective for treatment of retinal diseases due to corneal and conjunctival barriers, and rapid precorneal tear loss.^{34,35} Intravitreal and subconjunctival injections are invasive and carry risks of infection.^{12,36} In contrast, intranasal insulin administration is non-invasive, can be easily self-administered, avoids hepatic first-pass elimination and has been shown to reach the central nervous system within minutes without raising peripheral insulin or causing hypoglycemia.^{13,14}

Insulin delivered intranasally has been shown to cross the blood brain barrier and was detected in the brainstem, cerebellum, substantia nigra/ventral tegmental area, olfactory bulb, striatum, hippocampus and thalamus/hypothalamus.³⁷ In experimental models of multiple sclerosis and traumatic optic neuropathy, intranasally administered amnion cell secretome have been found at therapeutic levels in the optic nerve and retina.^{38,39}

In various aspects described herein are methods of treating or preventing one or more symptoms associated with eye disease, in particular diabetic retinopathy, in a mammal in need thereof via intranasal delivery of insulin or an insulin analog or derivative.

Further disclosed are a variety of pharmaceutical formulations, kits, and dosage forms for carrying out the methods of intranasal delivery of an effective amount of insulin (or an insulin analog or insulin derivative) to a mammal in need thereof to of treat or prevent one or more symptoms associated with eye disease, in particular diabetic retinopathy.

Other systems, methods, features, and advantages of the formulations and methods for treating and preventing diabetic retinopathy will be or become apparent to one with skill in the art upon examination of the following drawings and detailed description. It is intended that all such additional systems, methods, features, and advantages be included within this description, be within the scope of the present disclosure, and be protected by the accompanying claims.

Although any methods and materials similar or equivalent to those described herein can also be used in the practice or testing of the present disclosure, the preferred methods and materials are now described. Functions or constructions well-known in the art may not be described in detail for brevity and/or clarity. Aspects of the present disclosure will employ, unless otherwise indicated, techniques of biotechnology, chemistry, pharmacology, ophthalmology, and the like, which are within the skill of the art. Such techniques are explained fully in the literature.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. Furthermore, any incorporation by reference of patents and patent applications to which the instant application claims priority is not intended to extend to any lexicographical definitions in the patents and patent applications so incorporated and should not be read as limiting the accompanying claims.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method may be described in a particular order in the specification, other logical ordering of the steps is intended to also be covered and may be recited in the claims.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and the range less than ‘y’. The range can also be expressed as an upper limit e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some aspects, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in aspects of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

As used herein, the term “and/or” includes any and all combinations of one or more of the associated listed items. Expressions such as “at least one of,” when preceding a list of elements, modify the entire list of elements and do not modify the individual elements of the list.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

“Effective amount” means an amount of insulin (or an insulin analog or insulin derivative) effective in producing the desired therapeutic effect, whether in preventing the onset of eye disease or one or more symptoms associated with eye disease or in slowing or stopping the progression of eye disease or one or more symptoms of eye disease. In some instances, the effective amount is effective at reversing one or more symptoms associated with the eye disease. For example, the effective amount may be effective at slowing or reversing one or more symptoms of diabetic retinopathy when administered to a mammal in need thereof.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, troubleshooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a disorder of uncontrolled cellular proliferation, a disorder associated with a LCK kinase dysfunction, and/or an immunologic disease or pathological condition involving an immunologic component. The effect can be therapeutic in terms of a partial or complete cure of a disease, condition, symptom or adverse effect attributed to the disease, disorder, or condition. The term “treatment” as used herein can include any treatment of eye disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Methods of Treating Eye Disease

In various aspects, methods are provided for treating eye disease in a subject in need thereof. The eye disease can include one or more of ischemic optic neuropathy, non-ischemic optic neuropathy, macular degeneration, retinal degeneration, retinal detachment, non-diabetic retinopathies, retinal arterial occlusion, retinal vein occlusion, primary glaucomas, secondary glaucomas. primary cataracts, secondary cataracts, orbital disease, corneal disease, keratopathies, or corneal dystrophies. In some aspects, the eye disease is associated with diabetes in the subject. For example, the eye disease can include diabetic retinopathy, betic macular edema, cataracts, glaucoma, or a combination thereof.

### Diabetic Eye Disease

In some aspects, the methods described herein include administering intranasally an insulin, insulin analog, or insulin derivative to a subject to treat a diabetic eye disease. Diabetic eye diseases encompass a diverse group of ocular disorders that arise as a result of diabetes mellitus and its associated metabolic abnormalities. Standard progression of diabetic eye diseases varies depending on the specific condition and the duration and severity of diabetes. Symptoms experienced by individuals with diabetic eye diseases reflect the affected structures of the eye, including the retina, lens, and blood vessels. Common symptoms include blurred vision, floaters (spots or cobweb-like shapes in the visual field), vision loss, and alterations in color perception. The severity and course of the diseases depend on the specific condition and the control of diabetes.

The treatment of diabetic eye diseases conventionally involves a multidisciplinary approach aimed at controlling diabetes, managing ocular complications, and preserving visual function. Therapeutic options may include strict glycemic control through diet, exercise, oral antidiabetic medications, or insulin therapy. Ocular interventions may include laser photocoagulation to treat diabetic retinopathy, intravitreal injections of anti-vascular endothelial growth factor (VEGF) agents for macular edema, or surgical procedures such as vitrectomy for advanced cases. Regular monitoring of blood glucose levels, comprehensive eye examinations, and timely intervention are crucial in managing diabetic eye diseases.

In some aspects, the methods described herein include treating a subject having a diabetic eye disease with a combination of intranasal insulin (or an insulin analog or derivative) in combination with convention diabetes interventions such as glycemic control through diet, exercise, oral antidiabetic medications, or conventional insulin therapy. In some aspects, the subject has diabetes but has not yet developed a diabetic eye disease. In such instances a lower prophylactic dosage may be administered intranasally to prevent or slow the onset for a diabetic eye disease. Diabetic eye diseases can include any disease or condition effecting the vision or eyes of subjects having diabetes. For example, the eye disease can include diabetic retinopathy, diabetic macular edema, cataracts, glaucoma, or a combination thereof.

The progression of diabetic eye diseased described herein can be monitored through various methods to assess the severity of the disease and guide treatment decisions.

Dilated Fundus Examination is a standard procedure in which the ophthalmologist examines the retina after dilating the patient's pupils. Using specialized instruments, the doctor evaluates the presence of characteristic retinal changes, such as microaneurysms, hemorrhages, exudates, and abnormal blood vessels. The examination helps determine the stage and severity of diabetic retinopathy.

Optical Coherence Tomography (OCT) is a non-invasive imaging technique that provides detailed cross-sectional images of the retina. It allows visualization of retinal thickness, macular edema (swelling), and the integrity of retinal layers. OCT helps detect fluid accumulation, cysts, and other structural changes associated with diabetic retinopathy.

Fluorescein Angiography (FA) involves injecting a fluorescent dye into the patient's bloodstream, which highlights the blood vessels in the retina. The ophthalmologist captures images of the dye as it circulates through the retina using specialized cameras. FA helps identify areas of abnormal blood vessel growth (neovascularization), leakage, and areas of ischemia (lack of blood flow) in the retina.

Indocyanine Green Angiography (ICG), which is similar to FA, uses a different dye called indocyanine green to visualize the choroidal blood vessels, which lie beneath the retina. It provides additional information about the blood flow in the deeper layers of the retina and choroid, aiding in the assessment of certain forms of diabetic retinopathy.

High-resolution retinal photography captures detailed images of the retina, allowing for the documentation of retinal changes over time. These photographs serve as a reference for monitoring disease progression and evaluating the effectiveness of treatment.

Microperimetry assesses the sensitivity of the central retina by measuring the patient's ability to detect and respond to visual stimuli. This test helps evaluate macular function and identify any changes associated with macular edema.

Visual Acuity Testing: Visual acuity is measured using an eye chart to assess central vision and detect any changes over time. It is a subjective measure that indicates the patient's ability to see details and read at a distance. Regular visual acuity assessments help monitor changes in vision and determine the impact of cataracts on visual function.

Slit-Lamp Examination: Slit-lamp examination is a specialized examination technique that allows the eye care professional to examine the anterior segment of the eye, including the lens. With the help of a biomicroscope and a slit of light, the ophthalmologist or optometrist can evaluate the degree of lens opacification and assess the progression of cataracts.

Ophthalmoscopy: Ophthalmoscopy, also known as fundoscopy, involves the use of an ophthalmoscope to examine the internal structures of the eye, including the lens. The doctor can visualize the lens to determine the presence and severity of cataracts.

Contrast Sensitivity Testing: Contrast sensitivity measures the patient's ability to distinguish between objects with varying levels of contrast. This test assesses the visual system's ability to perceive details and changes in contrast. Serial contrast sensitivity testing helps monitor changes in visual function caused by cataracts.

In some aspects, the methods include monitoring a subject receiving intranasal insulin delivery using one or more of Dilated Fundus Examination, OCT, FA, ICG, high-resolution retinal photography, visual acuity testing, slit-lamp examination, ophthalmoscopy, contrast sensitivity testing, electroretinogram, or a combination thereof. In some aspects, the methods provided herein result in a reduction of the progression of or a reduction in the presence of characteristic retinal changes, such as microaneurysms, hemorrhages, exudates, and abnormal blood vessels. In some aspects, the methods provided herein result in a reduction of the progression of or a reduction in the presence of fluid accumulation, cysts, and other structural changes associated with diabetic retinopathy. In some aspects, the methods described herein result in a reduction in the progression of or a reduction in the presence of neovascularization, leakage, and areas of ischemia in the retina. In some aspects, the methods described herein help to prevent a flattening of the b wave in the electroretinogram as measured over the course of treatment for a patient with diabetic retinopathy or at risk for diabetic retinopathy.

### Diabetic Retinopathy

Diabetic retinopathy is a progressive ocular disorder that develops as a complication of diabetes mellitus, primarily affecting the blood vessels in the retina. Standard progression of diabetic retinopathy varies depending on the stage of the disease and the duration and control of diabetes. Symptoms experienced by individuals with diabetic retinopathy reflect the extent of retinal damage and may include blurred vision, fluctuating vision, floaters (spots or cobweb-like shapes in the visual field), and eventually vision loss. The severity and course of the disease depend on various factors, including blood glucose control, blood pressure, and individual susceptibility.

The treatment of diabetic retinopathy conventionally involves a multifaceted approach aimed at managing risk factors, controlling diabetes, and preventing or treating complications. Therapeutic interventions may include strict glycemic control through lifestyle modifications, oral antidiabetic medications, or insulin therapy. Blood pressure and lipid management are also crucial in slowing the progression of retinal damage. Ophthalmic interventions may include laser photocoagulation to seal leaking blood vessels or reduce abnormal vessel growth, intravitreal injections of anti-vascular endothelial growth factor (VEGF) agents to manage macular edema or proliferative diabetic retinopathy, and vitrectomy for advanced cases with vitreous hemorrhage or tractional retinal detachment. Regular eye examinations, including dilated fundus examinations, are essential for timely detection and treatment of diabetic retinopathy. Methods described herein can include treating a subject having diabetic retinopathy via intranasal administration of insulin or an insulin analog or derivative. The method can include co-administering insulin or an insulin analog or derivative along with conventional treatments for diabetic retinopathy such as laser photocoagulation to seal leaking blood vessels or reduce abnormal vessel growth, intravitreal injections of anti-vascular endothelial growth factor (VEGF) agents to manage macular edema or proliferative diabetic retinopathy. In some instances the subject is a diabetic and also receiving one or more treatments for diabetes including oral antidiabetic medications, or oral insulin therapy.

### Diabetic Macular Edema

Diabetic macular edema is a vision-threatening complication of diabetic retinopathy characterized by the accumulation of fluid in the macula, the central part of the retina responsible for sharp vision. Standard progression of diabetic macular edema varies depending on the severity of retinal vascular changes and the duration of diabetes. Symptoms experienced by individuals with diabetic macular edema include blurred or distorted central vision, difficulty reading, and changes in color perception. The severity and course of the disease depend on factors such as the extent of retinal involvement and the control of diabetes.

The treatment of diabetic macular edema conventionally involves a multifaceted approach aimed at reducing macular edema, restoring macular function, and preserving visual acuity. Therapeutic options may include the use of intravitreal injections of anti-vascular endothelial growth factor (VEGF) agents to reduce vascular permeability and edema, thereby improving macular anatomy and visual acuity. Other treatments may include corticosteroid injections or implants to reduce inflammation and macular edema. Laser photocoagulation, particularly focal or grid laser, may be employed to target and seal leaking blood vessels in the macula. Additionally, optimizing systemic control of diabetes through lifestyle modifications, medication, or insulin therapy is crucial in managing diabetic macular edema.

Methods described herein can include treating a subject having diabetic macular edema via intranasal administration of insulin or an insulin analog or derivative. The method can include co-administering insulin or an insulin analog or derivative along with conventional treatments for diabetic macular edema such as intravitreal injections of anti-vascular endothelial growth factor (VEGF) agents to reduce vascular permeability and edema, corticosteroid injections or implants to reduce inflammation and macular edema, laser photocoagulation, particularly focal or grid laser, to target and seal leaking blood vessels. In some instances the subject is a diabetic and also receiving one or more treatments for diabetes including oral antidiabetic medications, or oral insulin therapy.

### Cataracts

Diabetic cataracts are a common complication of diabetes mellitus characterized by the clouding of the eye's natural lens. Standard progression of diabetic cataracts varies depending on the duration and control of diabetes, as well as individual susceptibility to the condition. Symptoms experienced by individuals with diabetic cataracts include blurred or hazy vision, increased sensitivity to glare, and difficulties with night vision. The severity and course of the cataracts depend on various factors, including the level of glycemic control, age, and other comorbidities.

The treatment of diabetic cataracts conventionally involves a comprehensive approach aimed at managing diabetes, optimizing glycemic control, and addressing visual impairment. Therapeutic interventions may include lifestyle modifications, oral antidiabetic medications, or insulin therapy to achieve and maintain target blood glucose levels. Cataract surgery, the primary treatment for cataracts, involves removing the clouded natural lens and replacing it with an artificial intraocular lens (IOL). The timing of cataract surgery is determined based on the degree of visual impairment and the patient's overall ocular health. Postoperative care includes regular follow-up visits and management of any complications.

Methods described herein can include treating a subject having diabetic cataracts via intranasal administration of insulin or an insulin analog or derivative. In some instances the subject is a diabetic and also receiving one or more treatments for diabetes including oral antidiabetic medications, or oral insulin therapy. In some instances, the patient is a diabetic and the methods prevent the onset or slow the progression of cataracts in the subject, which can eliminate or reduce the risk of the patient requiring cataract surgery.

### Glaucoma

Glaucoma caused by diabetes, known as diabetic glaucoma, is a group of progressive eye diseases characterized by damage to the optic nerve and loss of peripheral vision. Standard progression of diabetic glaucoma varies depending on the specific type of glaucoma, the severity of the disease, and the control of diabetes. Symptoms experienced by individuals with diabetic glaucoma include a gradual loss of vision, peripheral vision impairment or “tunnel vision,” eye pain, and the presence of optic nerve abnormalities. The severity and course of the disease depend on various factors, including the type and stage of glaucoma, intraocular pressure levels, and the control of diabetes.

The treatment of diabetic glaucoma conventionally involves a comprehensive approach aimed at lowering intraocular pressure, preserving optic nerve function, and preventing further visual deterioration. Therapeutic interventions may include the use of topical or oral medications to reduce intraocular pressure, such as prostaglandin analogs, beta-blockers, or carbonic anhydrase inhibitors. Laser trabeculoplasty or incisional surgeries, such as trabeculectomy or drainage device implantation, may be considered to enhance aqueous humor outflow and lower intraocular pressure. Additionally, strict control of diabetes through lifestyle modifications, medication, or insulin therapy is crucial in managing diabetic glaucoma.

### Other Eye Diseases

Ischemic optic neuropathy is a medical condition characterized by insufficient blood supply to the optic nerve, leading to optic nerve damage and subsequent visual impairments. Standard progression of ischemic optic neuropathy typically involves an acute or subacute onset of symptoms, with patients experiencing sudden or gradual vision loss, often occurring in one eye. The visual impairment may manifest as blurred vision, decreased visual acuity, or a loss of peripheral vision. In severe cases, complete vision loss in the affected eye may occur. The treatment of ischemic optic neuropathy conventionally focuses on addressing the underlying causes, improving blood circulation, reducing inflammation, and preserving remaining vision. Current therapeutic approaches involve the administration of vasodilators to enhance blood flow, anti-inflammatory agents to reduce inflammation, and neuroprotective compounds to support optic nerve health and regeneration. Additionally, lifestyle modifications, such as smoking cessation and blood pressure management, may be recommended to mitigate further damage to the optic nerve. Methods described herein can include treating a subject having ischemic optic neuropathy via intranasal administration of insulin or an insulin analog or derivative. The method can include co-administering insulin or an insulin analog or derivative along with conventional treatments for ischemic optic neuropathy such as asodilators to enhance blood flow, anti-inflammatory agents to reduce inflammation, and neuroprotective compounds to support optic nerve health and regeneration.

Non-ischemic optic neuropathy is a medical condition characterized by optic nerve damage resulting from causes other than insufficient blood flow, such as inflammation, compression, toxic exposure, or hereditary factors, leading to visual impairments. Standard progression of non-ischemic optic neuropathy encompasses a wide range of etiologies, with variable symptomatology among patients. Common symptoms include gradual or sudden vision loss, changes in color vision, visual field defects, optic nerve abnormalities, or a combination of these manifestations. The treatment of non-ischemic optic neuropathy conventionally focuses on identifying and addressing the underlying causes, reducing inflammation, and optimizing optic nerve function. Treatment modalities often involve targeted therapies specific to the etiology, such as anti-inflammatory agents, immunosuppressive drugs, or surgical interventions to alleviate compressive factors. Neuroprotective compounds may also be administered to enhance optic nerve health and support regeneration. Methods described herein can include treating a subject having non-ischemic optic neuropathy via intranasal administration of insulin or an insulin analog or derivative. The method can include co-administering insulin or an insulin analog or derivative along with conventional treatments for non-ischemic optic neuropathy such as anti-inflammatory agents, immunosuppressive drugs, or surgical interventions.

Macular degeneration is a medical condition characterized by progressive damage to the macula, leading to central vision impairment or loss. Standard progression of macular degeneration encompasses different stages, including early, intermediate, and advanced stages. In the early and intermediate stages, patients may be asymptomatic or experience mild visual changes, such as blurry vision or distortion in the central visual field. As the condition advances, symptoms become more pronounced, with individuals experiencing significant visual impairment. Symptoms include blurred or distorted central vision, dark or empty areas in the central visual field (scotomas), and difficulty reading or recognizing faces. The treatment of macular degeneration conventionally aims to slow disease progression, preserve existing vision, and prevent further visual deterioration. Current therapeutic approaches involve the administration of anti-angiogenic drugs to inhibit abnormal blood vessel growth in the macula, photodynamic therapy to selectively destroy abnormal blood vessels, and the use of intraocular injections of corticosteroids to reduce inflammation and swelling. Lifestyle modifications, such as nutritional supplements, regular eye examinations, and low vision aids, may also be recommended to manage the condition. Methods described herein can include treating a subject having macular degeneration via intranasal administration of insulin or an insulin analog or derivative. The method can include co-administering insulin or an insulin analog or derivative along with conventional treatments for macular degeneration such as the administration of anti-angiogenic drugs to inhibit abnormal blood vessel growth in the macula, photodynamic therapy to selectively destroy abnormal blood vessels, and the use of intraocular injections of corticosteroids to reduce inflammation and swelling.

Retinal degeneration encompasses a group of progressive conditions characterized by the deterioration of the retinal tissue, leading to visual impairment or loss. Standard progression of retinal degeneration involves the gradual development and worsening of symptoms. Patients typically experience decreased visual acuity, impaired color vision, and visual field defects. As the condition advances, individuals may also develop night blindness and difficulties with central and peripheral vision. The treatment of retinal degeneration conventionally aims to slow disease progression, preserve existing vision, and promote retinal tissue health. Current therapeutic approaches include the administration of neuroprotective compounds to support the survival of retinal cells, the use of gene therapy to replace or repair defective genes associated with retinal degeneration, and the implantation of retinal prostheses to bypass damaged retinal cells and stimulate remaining functional cells. Additionally, lifestyle modifications, such as the use of low vision aids and visual rehabilitation programs, may be recommended to optimize visual function and improve quality of life. Methods described herein can include treating a subject having retinal degeneration via intranasal administration of insulin or an insulin analog or derivative. The method can include co-administering insulin or an insulin analog or derivative along with conventional treatments for retinal degeneration such as administration of neuroprotective compounds to support the survival of retinal cells, the use of gene therapy to replace or repair defective genes associated with retinal degeneration, and the implantation of retinal prostheses to bypass damaged retinal cells and stimulate remaining functional cells.

Retinal detachment is a condition characterized by the separation of the neurosensory retina from the underlying retinal pigment epithelium, resulting in vision loss if left untreated. Standard progression of retinal detachment involves distinct stages and associated symptoms. Initially, patients may experience sudden or gradual vision loss, often described as a shadow or curtain obstructing part of the visual field. Additionally, individuals may perceive floaters, which appear as spots or specks drifting across the visual field, and flashes of light, resembling brief bursts of illumination. The treatment of retinal detachment conventionally aims to reattach the detached retina and prevent further vision deterioration. Current therapeutic approaches include surgical procedures such as scleral buckling, vitrectomy, and pneumatic retinopexy. Scleral buckling involves the placement of a silicone band around the eye to provide external support and reposition the detached retina. Vitrectomy involves the removal of the vitreous gel from the eye and subsequent filling with a gas or silicone oil to reattach the retina. Pneumatic retinopexy utilizes the injection of a gas bubble into the eye, positioning it strategically to push the detached retina back into place. These techniques are often combined with laser photocoagulation or cryotherapy to seal retinal tears and prevent further detachment. Methods described herein can include treating a subject having retinal detachment via intranasal administration of insulin or an insulin analog or derivative. The method can include co-administering insulin or an insulin analog or derivative along with conventional treatments for retinal detachment such as surgical procedures such as scleral buckling, vitrectomy, and pneumatic retinopexy.

Non-diabetic retinopathies encompass a group of retinal disorders characterized by pathological changes in the blood vessels and tissues of the retina, occurring in the absence of diabetes mellitus. Standard progression of non-diabetic retinopathies involves distinct stages and associated symptoms. Initially, patients may experience visual disturbances, such as blurred or distorted vision, and the perception of floaters-dark spots or cobweb-like structures drifting across the visual field. Decreased visual acuity and difficulty seeing in dim light may also be observed. As the condition progresses, retinal hemorrhages, leakage of fluid and lipids (exudation), macular edema (swelling of the central part of the retina), and in severe cases, neovascularization (abnormal blood vessel growth) and retinal detachment may occur, leading to significant visual impairment. The treatment of non-diabetic retinopathies conventionally aims to manage the underlying causes, reduce inflammation, and preserve or improve visual function. Current therapeutic approaches include the administration of anti-angiogenic agents to inhibit abnormal blood vessel growth, corticosteroids to reduce inflammation and edema, and laser photocoagulation or intravitreal injections to target specific lesions or areas of neovascularization. Additionally, lifestyle modifications, such as blood pressure management, may be recommended to mitigate further damage to the retinal blood vessels. Methods described herein can include treating a subject having non-diabetic retinopathies via intranasal administration of insulin or an insulin analog or derivative. The method can include co-administering insulin or an insulin analog or derivative along with conventional treatments for non-diabetic retinopathies such as the administration of anti-angiogenic agents to inhibit abnormal blood vessel growth, corticosteroids to reduce inflammation and edema, and laser photocoagulation or intravitreal injections to target specific lesions or areas of neovascularization.

Retinal arterial occlusion is a medical condition characterized by the blockage of the retinal artery, leading to interrupted blood flow and subsequent retinal ischemia. Standard progression of retinal arterial occlusion involves an acute onset of symptoms. Patients typically experience sudden, painless vision loss in the affected eye, often described as a curtain or shadow obscuring part or all of the visual field. Visual acuity may be severely affected, and individuals may also notice visual field defects or color vision abnormalities. The treatment of retinal arterial occlusion conventionally aims to restore blood flow, preserve vision, and prevent further complications. Current therapeutic approaches include the administration of vasodilators to improve blood circulation, antiplatelet agents to prevent clot formation, and neuroprotective compounds to support retinal tissue health. Additional treatments may include intraocular pressure-lowering medications, hyperbaric oxygen therapy, and interventions to address underlying cardiovascular risk factors. Timely intervention is crucial to maximize the chances of vision recovery and prevent permanent damage to the retina. Methods described herein can include treating a subject having retinal arterial occlusion via intranasal administration of insulin or an insulin analog or derivative. The method can include co-administering insulin or an insulin analog or derivative along with conventional treatments for retinal arterial occlusion such as the administration of vasodilators to improve blood circulation, antiplatelet agents to prevent clot formation, and neuroprotective compounds to support retinal tissue health.

Retinal vein occlusion is a medical condition characterized by the blockage of a retinal vein, leading to impaired venous blood flow and subsequent retinal ischemia. Standard progression of retinal vein occlusion involves an acute or subacute onset of symptoms. Patients typically experience sudden, painless vision loss in the affected eye, often accompanied by blurred or distorted vision. Visual acuity may be significantly affected, and individuals may notice visual field defects, color vision abnormalities, or the presence of floaters. The treatment of retinal vein occlusion conventionally aims to improve blood flow, reduce macular edema, and preserve or improve visual function. Current therapeutic approaches include the administration of anti-vascular endothelial growth factor (anti-VEGF) agents to reduce macular edema and promote retinal perfusion, corticosteroids to decrease inflammation and edema, and laser photocoagulation to address retinal neovascularization and complications. In some cases, intraocular pressure-lowering medications may be prescribed to manage associated glaucoma. Timely intervention is crucial to prevent further vision loss and mitigate potential long-term complications. Methods described herein can include treating a subject having retinal vein occlusion via intranasal administration of insulin or an insulin analog or derivative. The method can include co-administering insulin or an insulin analog or derivative along with conventional treatments for retinal vein occlusion such as the administration of anti-vascular endothelial growth factor (anti-VEGF) agents to reduce macular edema and promote retinal perfusion, corticosteroids to decrease inflammation and edema, and laser photocoagulation to address retinal neovascularization and complications.

Primary glaucomas are chronic eye diseases characterized by progressive optic nerve damage and visual field loss, occurring in the absence of other ocular or systemic conditions. Standard progression of primary glaucomas involves an insidious onset, often with no noticeable symptoms in the early stages, making timely diagnosis challenging. As the disease progresses, patients may experience gradual peripheral vision loss, resulting in tunnel vision, difficulty with night vision, and blurred vision. In advanced cases, complete vision loss can occur. The treatment of primary glaucomas conventionally aims to reduce intraocular pressure (IOP), prevent further optic nerve damage, and preserve visual function. Current therapeutic approaches include the administration of topical or systemic medications to lower IOP, such as prostaglandin analogs, beta-blockers, carbonic anhydrase inhibitors, or alpha-2 adrenergic agonists. Laser trabeculoplasty and surgical interventions, such as trabeculectomy or glaucoma drainage devices, may be utilized to enhance aqueous humor drainage and further reduce IOP. Regular monitoring and assessment of optic nerve health, visual field testing, and lifestyle modifications, such as maintaining a healthy lifestyle and managing systemic factors contributing to glaucoma progression, are also essential components of management. Methods described herein can include treating a subject having primary glaucomas via intranasal administration of insulin or an insulin analog or derivative. The method can include co-administering insulin or an insulin analog or derivative along with conventional treatments for primary glaucomas such as the administration of topical or systemic medications to lower IOP, such as prostaglandin analogs, beta-blockers, carbonic anhydrase inhibitors, or alpha-2 adrenergic agonists.

Secondary glaucomas encompass a diverse group of eye conditions characterized by increased intraocular pressure (IOP) and optic nerve damage that arise as a consequence of identifiable underlying causes or associated ocular or systemic conditions. Standard progression of secondary glaucomas varies depending on the specific etiology, with symptoms typically reflecting elevated IOP and optic nerve compromise. Patients may experience gradual or sudden vision loss, eye pain, redness, headache, and visual field defects. The severity of symptoms and the rate of disease progression are influenced by the nature of the underlying cause. The treatment of secondary glaucomas conventionally aims to address the underlying cause, reduce IOP, and preserve visual function. Current therapeutic approaches involve a multifaceted approach tailored to the specific etiology of the condition. Treatments may include topical or systemic medications to lower IOP, laser therapies such as trabeculoplasty or cyclophotocoagulation, and surgical interventions such as filtration surgery, drainage devices, or cyclodestructive procedures. Additionally, management of the underlying condition or associated systemic factors contributing to glaucoma progression may be necessary. Regular monitoring of IOP, optic nerve health assessment, and visual field testing are crucial in managing secondary glaucomas. Methods described herein can include treating a subject having secondary glaucomas via intranasal administration of insulin or an insulin analog or derivative. The method can include co-administering insulin or an insulin analog or derivative along with conventional treatments for secondary glaucomas such as systemic medications to lower IOP, laser therapies such as trabeculoplasty or cyclophotocoagulation, and surgical interventions such as filtration surgery, drainage devices, or cyclodestructive procedures.

Primary cataracts are characterized by the opacification or clouding of the natural crystalline lens of the eye and are typically associated with aging or genetic factors. Standard progression of primary cataracts involves a gradual development of symptoms. Patients may experience blurry or cloudy vision, reduced visual acuity, decreased color perception, increased sensitivity to glare, and difficulties with night vision. The severity of symptoms and the rate of cataract progression vary among individuals. The treatment of primary cataracts conventionally involves surgical intervention known as cataract surgery. The goal of cataract surgery is to remove the clouded lens and replace it with an artificial intraocular lens (IOL), restoring clear vision. Cataract surgery techniques include phacoemulsification, where the cloudy lens is emulsified and removed through a small incision, and extracapsular cataract extraction, where the lens is removed intact. Following lens removal, an IOL is implanted to replace the natural lens, allowing for visual rehabilitation. Advanced technologies, such as femtosecond laser-assisted cataract surgery and the use of premium IOLs, offer improved precision and options for personalized visual outcomes. Methods described herein can include treating a subject having primary cataracts via intranasal administration of insulin or an insulin analog or derivative. The method can include co-administering insulin or an insulin analog or derivative along with conventional treatments for primary cataracts such as surgical intervention.

Secondary cataracts are characterized by the opacification or clouding of the natural crystalline lens of the eye and occur as a result of various underlying conditions or factors. Standard progression of secondary cataracts varies depending on the specific etiology, with symptoms typically reflecting the underlying cause and lens opacification. Patients may experience blurry or cloudy vision, reduced visual acuity, glare sensitivity, and difficulties with color perception. The severity and rate of progression depend on the underlying condition and its impact on lens clarity. The treatment of secondary cataracts conventionally involves cataract surgery, similar to primary cataracts. The objective is to remove the clouded lens and replace it with an artificial intraocular lens (IOL), restoring clear vision. Cataract surgery techniques include phacoemulsification or extracapsular cataract extraction, followed by IOL implantation. The selection of the appropriate IOL depends on factors such as the patient's visual needs, potential comorbidities, and surgeon preference. Advanced technologies, such as femtosecond laser-assisted cataract surgery and premium IOL options, enhance surgical precision and offer personalized visual outcomes. Methods described herein can include treating a subject having secondary cataracts via intranasal administration of insulin or an insulin analog or derivative. The method can include co-administering insulin or an insulin analog or derivative along with conventional treatments for secondary cataracts such as surgical intervention.

Orbital diseases encompass a diverse group of conditions affecting the structures within the orbital cavity. Standard progression of orbital diseases varies depending on the specific etiology and affected structures. Patients may experience a range of symptoms reflecting the underlying cause and extent of involvement. Common symptoms include pain, swelling, proptosis (forward displacement of the eyeball), diplopia (double vision), and visual disturbances. The severity and course of the disease depend on the specific condition and its impact on orbital structures. The treatment of orbital diseases conventionally involves a multifaceted approach tailored to the underlying cause and clinical manifestations. Depending on the specific condition, treatment options may include medical interventions such as corticosteroids to reduce inflammation, immunosuppressive agents for autoimmune conditions, antimicrobial therapy for infectious processes, or targeted therapies for neoplastic disorders. Surgical interventions may be necessary to address structural abnormalities, relieve pressure on the optic nerve, or remove tumors or cysts. Visual rehabilitation, ocular lubrication, and management of associated symptoms are important components of care. Methods described herein can include treating a subject having orbital diseases via intranasal administration of insulin or an insulin analog or derivative. The method can include co-administering insulin or an insulin analog or derivative along with conventional treatments for orbital diseases such as corticosteroids to reduce inflammation, immunosuppressive agents for autoimmune conditions, antimicrobial therapy for infectious processes, or targeted therapies for neoplastic disorders.

Corneal diseases encompass a diverse group of conditions affecting the cornea, the clear front part of the eye that covers the iris and pupil. Standard progression of corneal diseases varies depending on the specific etiology and the layers or structures of the cornea that are affected. Patients may experience a variety of symptoms reflecting the underlying cause and the extent of corneal involvement. Common symptoms include pain, redness, blurred vision, tearing, and sensitivity to light. The severity and course of the disease depend on the specific condition and its impact on corneal health. The treatment of corneal diseases conventionally involves a multidisciplinary approach tailored to the underlying cause and the specific characteristics of the condition. Depending on the nature of the disease, treatment options may include the use of topical or systemic medications to reduce inflammation, control infection, or promote corneal healing. Surgical interventions such as corneal transplantation, corneal cross-linking, or laser procedures may be necessary to restore corneal integrity and visual function. Supportive therapies such as the use of artificial tears, bandage contact lenses, or therapeutic contact lenses may also be employed to alleviate symptoms and promote corneal healing. Methods described herein can include treating a subject having corneal diseases via intranasal administration of insulin or an insulin analog or derivative. The method can include co-administering insulin or an insulin analog or derivative along with conventional treatments for corneal diseases such as the use of topical or systemic medications to reduce inflammation, control infection, or promote corneal healing.

Keratopathies encompass a diverse group of corneal disorders characterized by abnormalities in the structure, function, or clarity of the cornea. Standard progression of keratopathies varies depending on the specific etiology and the pathophysiological mechanisms involved. Patients with keratopathies may experience a range of symptoms reflecting the affected layers and structures of the cornea. Common symptoms include blurred vision, pain, redness, tearing, and sensitivity to light. The severity and course of the disease depend on the specific condition and its impact on corneal health. The treatment of keratopathies conventionally involves a multidimensional approach tailored to the underlying cause and clinical manifestations. Depending on the nature of the disorder, treatment options may include the use of topical or systemic medications to reduce inflammation, manage infection, or promote corneal healing. Surgical interventions such as corneal transplantation, keratoplasty, or phototherapeutic keratectomy may be employed to restore corneal integrity and visual function. Supportive therapies such as the use of artificial tears, therapeutic contact lenses, or amniotic membrane transplantation may also be utilized to alleviate symptoms and facilitate corneal healing. Methods described herein can include treating a subject having keratopathies via intranasal administration of insulin or an insulin analog or derivative. The method can include co-administering insulin or an insulin analog or derivative along with conventional treatments for keratopathies such as use of topical or systemic medications to reduce inflammation, manage infection, or promote corneal healing.

Corneal dystrophies encompass a diverse group of inherited disorders characterized by progressive changes in the structure and function of the cornea. Standard progression of corneal dystrophies varies depending on the specific subtype and the genetic mutation involved. Symptoms experienced by individuals with corneal dystrophies reflect the affected layers and structures of the cornea. Common symptoms include blurred vision, pain, photophobia (sensitivity to light), and the presence of corneal opacities. The severity and course of the disease depend on the specific subtype and its impact on corneal health. The treatment of corneal dystrophies conventionally focuses on managing symptoms, slowing disease progression, and preserving visual function. Therapeutic options may include the use of topical medications to alleviate symptoms, such as lubricating eye drops for dryness or specialized ointments for corneal erosions. Surgical interventions, such as corneal transplantation or phototherapeutic keratectomy, may be considered in cases where vision is significantly compromised or when corneal opacities impair visual acuity. Additionally, genetic counseling and testing play a crucial role in providing patients and families with information about disease progression and facilitating appropriate management strategies. Methods described herein can include treating a subject having corneal dystrophies via intranasal administration of insulin or an insulin analog or derivative. The method can include co-administering insulin or an insulin analog or derivative along with conventional treatments for corneal dystrophies such as the use of topical medications to alleviate symptoms, such as lubricating eye drops for dryness or specialized ointments for corneal erosions.

### Insulin and Insulin Analogs and Derivatives

The methods and formulations include insulin or an insulin derivative or analog. Unless context dictates otherwise, references to insulin in the specification shall be interpreted to include references to insulin derivatives and insulin analogs. For example, where the specification describes methods of formulating insulin, such description should be understood to also encompass methods of formulating insulin analogs and insulin derivatives.

Insulin Lispro is a rapid-acting insulin analog that exhibits a faster onset of action and shorter duration compared to regular human insulin. Insulin Aspart is another rapid-acting insulin analog that provides a rapid onset of action and shorter duration by replacing proline with aspartic acid at the B28 position. Insulin Glulisine, also a rapid-acting insulin analog, has substitutions of lysine with glutamic acid at the B3 and B29 positions, enabling a rapid onset of action. Insulin Detemir, a long-acting insulin analog, forms soluble multihexamer complexes through a fatty acid chain attached to the B29 amino acid residue, resulting in a prolonged duration of action.

Insulin Glargine, a long-acting insulin analog, has a substitution of glycine with arginine at the A21 position and two additional arginine residues added to the B30 position, offering a prolonged duration of action with a relatively constant level of insulin. Insulin Degludec, an ultra-long-acting insulin analog, forms soluble multihexamer complexes through a fatty acid side chain attached to the B29 amino acid residue, providing an extended duration of action. Insulin Inhalation Powder is a unique form of insulin that is inhaled rather than injected, rapidly absorbed through the lungs.

Insulin Human refers to regular human insulin derived from recombinant DNA technology or extraction from animal sources. It is a short-acting insulin with an onset of action within 30 minutes. Insulin Regular is a short-acting insulin identical to human insulin, with an onset of action within 30 minutes and a duration of several hours. Insulin NPH (Neutral Protamine Hagedorn) is an intermediate-acting insulin that combines crystalline zinc insulin with protamine, resulting in a delayed onset and longer duration of action compared to regular insulin.

Insulin Lente is an intermediate-acting insulin formulation that combines regular insulin with zinc insulin, providing a faster onset than NPH insulin but a shorter duration of action. Insulin Ultralente is a long-acting insulin with a zinc suspension, offering a longer duration of action compared to NPH insulin. Insulin PZI (Protamine Zinc Insulin) is a long-acting insulin with protamine and zinc, providing a prolonged duration of action. Insulin Glucose Solution is a concentrated insulin solution used for intravenous infusion to maintain blood glucose levels.

Insulin Zinc Suspension is an intermediate-acting insulin formulation with a zinc suspension, resulting in a delayed onset and longer duration of action. Insulin Semilente is an intermediate-acting insulin similar to Lente insulin but with a shorter duration of action. Insulin Extended is a modified insulin formulation with extended duration of action, designed to minimize the frequency of injections. Insulin Biphasic is a mixed insulin formulation that combines rapid-acting and intermediate-acting insulins to provide both immediate and long-lasting blood glucose control.

Insulin Combinations refer to commercially available mixtures that combine different types of insulins in specific ratios. Examples include Humalog Mix 75/25 (75% insulin lispro protamine suspension and 25% insulin lispro) and NovoLog Mix 70/30 (70% insulin aspart protamine suspension and 30% insulin aspart). Lastly, Insulin Tregopil is an investigational oral insulin formulation that is being developed as an alternative to injectable insulin therapy.

### Intranasal Formulations

The insulin or insulin derivative can be formulated in an intranasal formulation for intranasal delivery. The intranasal formulation can include nasal sprays, nasal drops, nasal gels, nasal powders, nanoparticles, microemulsions, in situ gelling systems, or any other formulation suitable for delivering insulin or insulin derivatives intranasally.

### Nasal Sprays

The formulation can include nasal sprays. Nasal sprays are commonly used for intranasal drug delivery. These formulations typically consist of a solution or suspension of the therapeutic drug in a suitable vehicle, along with excipients and sometimes a propellant. The formulation is prepared by dissolving or suspending the drug in a solvent or vehicle, followed by the addition of appropriate excipients. The mixture is then homogenized and sterilized if required. Finally, the formulation is filled into suitable nasal spray containers with an appropriate delivery mechanism, such as a pump or a metered-dose spray.

Formulating insulin into a nasal spray involves several steps to ensure the stability. bioavailability, and effective delivery of the insulin through the nasal route. The process begins with the selection of excipients, including solvents, preservatives, pH adjusters, and viscosity modifiers. Insulin, preferably an insulin derivative with higher solubility like insulin lispro or insulin aspart, needs to be dissolved or suspended in a suitable solvent or vehicle. The pH of the formulation is adjusted to optimize insulin stability, often within a slightly acidic range. Preservatives may be added to prevent microbial growth, while viscosity modifiers help achieve appropriate spray characteristics and nasal retention time. The formulation is prepared by thoroughly mixing insulin and the selected excipients, ensuring uniform distribution and dissolved insulin particles. Sterilization techniques are applied to maintain the formulation's safety, and it is then filled into suitable nasal spray containers, such as metered-dose pumps, for controlled and accurate dosing during administration.

### Nasal Drops

The formulation can include nasal drops. Nasal drops involve the direct instillation of liquid medication into the nostrils. To prepare nasal drops, the therapeutic drug is dissolved or suspended in a suitable liquid vehicle, such as water or saline solution. The drug and vehicle are mixed thoroughly to ensure uniform distribution. The resulting solution or suspension is then filled into dropper bottles or pre-filled single-dose units under aseptic conditions.

To prepare insulin nasal drops, various factors need to be addressed. The selection of appropriate excipients is crucial to ensure insulin stability and enhance its absorption. Solvents like water or saline solution are typically used as the vehicle for dissolving or suspending insulin. The insulin derivative used, such as insulin lispro or insulin aspart, should have sufficient solubility in the chosen solvent.

The formulation process begins by dissolving or suspending insulin in the selected solvent or vehicle. This step can be facilitated by gentle heating or agitation, if needed. It is important to ensure complete dissolution or uniform suspension of insulin particles for consistent dosing.

To optimize stability and minimize the risk of microbial contamination, preservatives such as benzalkonium chloride or chlorhexidine can be added to the formulation. These preservatives help maintain the sterility of the nasal drops during use.

Once the insulin is dissolved or suspended, the formulation can be filled into suitable dropper bottles or pre-filled single-dose units under aseptic conditions. These containers should provide accurate dosing and metering.

### Nasal Gels

The formulation can include nasal gels. Nasal gels are semi-solid formulations that provide sustained drug release and improved drug retention in the nasal cavity. They are typically composed of a hydrogel or a mucoadhesive polymer. To prepare nasal gels, the polymer is first dispersed or dissolved in a suitable solvent or water. The drug is then incorporated into the polymer solution, followed by mixing and homogenization. The mixture is allowed to undergo gelation, either by cooling or by a chemical crosslinking reaction. The resulting gel is then filled into suitable containers or unit-dose applicators.

Formulating insulin into nasal gels involves a careful process to ensure stability, prolonged residence time, and controlled release of insulin in the nasal cavity. To prepare insulin nasal gels, several considerations should be addressed. The selection of appropriate polymers is crucial to create a gel matrix that can provide sustained release and mucoadhesive properties. Commonly used polymers include hydrogels or mucoadhesive polymers such as carbomers, cellulose derivatives, or chitosan.

The formulation process begins by dispersing or dissolving the selected polymer in a suitable solvent or water. Insulin, preferably an insulin derivative with higher solubility like insulin lispro or insulin aspart, is then incorporated into the polymer solution. The mixture is thoroughly mixed and homogenized to achieve uniform distribution of insulin within the gel matrix.

To optimize gel formation and stability, crosslinking agents or gelling enhancers can be added if necessary. These agents promote gelation, leading to the formation of a three-dimensional network within the gel matrix.

To enhance patient comfort and improve nasal application, the formulation can include appropriate additives such as buffers to adjust the pH and viscosity modifiers to achieve the desired gel consistency and ease of administration.

Once the insulin nasal gel is prepared, it can be filled into suitable containers or unit-dose applicators under aseptic conditions to maintain sterility.

### Nasal Powders

The formulation can include nasal powders. Nasal powders are dry formulations consisting of finely ground drug particles. The preparation of nasal powders typically involves milling or micronizing the drug to achieve the desired particle size. The drug particles are then mixed with suitable excipients, such as inert carriers or absorption enhancers, to improve powder flow and nasal absorption. The mixture is homogenized and filled into suitable nasal powder devices or containers.

Formulating insulin into nasal powders requires careful consideration to ensure stability, proper dispersion, and efficient nasal absorption. Here is a description of the process:

To prepare insulin nasal powders, several steps need to be followed. The first step involves obtaining a fine powder of insulin particles. This can be achieved through techniques such as milling or micronization, which reduce the size of insulin particles to enhance their dissolution and absorption.

Once the insulin particles are obtained, they are mixed with suitable excipients to improve powder flow and nasal absorption. Inert carriers such as lactose or mannitol are commonly used as diluents to ensure proper dispersion and consistent dosing. Absorption enhancers such as surfactants or absorption-promoting agents may also be included to enhance insulin absorption across the nasal mucosa.

The insulin powder formulation is thoroughly mixed to achieve a uniform distribution of insulin particles within the excipient matrix. Techniques such as blending or micronization can be employed to ensure proper mixing and homogeneity.

To facilitate the administration of nasal powders, suitable nasal delivery devices or insufflators can be used. These devices enable controlled and precise administration of the powder into the nasal cavity, ensuring proper deposition and absorption of the insulin particles.

### Nanoparticles

The formulation can include nanoparticles. Nanoparticle-based formulations for intranasal delivery involve encapsulating the therapeutic drug within nanoparticles made of polymers or lipids. The preparation of nanoparticles often includes techniques such as emulsion/solvent evaporation, nanoprecipitation, or nanoparticle self-assembly. These methods involve the preparation of a drug-polymer or drug-lipid solution, followed by the addition of a stabilizer and the formation of nanoparticles through techniques like sonication or high-pressure homogenization. The resulting nanoparticle suspension is then purified and concentrated before filling into suitable containers.

Formulating insulin into nanoparticles involves a complex process to achieve proper encapsulation and controlled release of the insulin. Here is a description of the general steps involved:

The formulation of insulin nanoparticles begins by selecting suitable polymers or lipids for the nanoparticle matrix. These materials should be biocompatible, capable of encapsulating insulin, and providing stability to the nanoparticles. Commonly used polymers include poly(lactic-co-glycolic acid) (PLGA) and chitosan, while lipids like phospholipids may also be utilized.

The process typically starts with the preparation of a polymer or lipid solution. Insulin, preferably in its soluble form such as insulin lispro or insulin aspart, is then added to the solution. Through techniques such as emulsion/solvent evaporation or nanoprecipitation, the insulin is encapsulated within the polymer or lipid matrix. This can involve emulsifying the polymer solution with the insulin solution or rapidly mixing them together to induce nanoparticle formation.

To stabilize the insulin nanoparticles and prevent aggregation, stabilizers such as surfactants or stabilizing agents can be added to the formulation. These agents help maintain the uniform dispersion of insulin within the nanoparticle matrix.

Once the nanoparticles are formed, they are typically subjected to purification steps such as centrifugation or filtration to remove any excess polymer or unencapsulated insulin. The purified insulin nanoparticles can then be concentrated to achieve the desired nanoparticle concentration.

To ensure sterility and long-term stability, the insulin nanoparticles may undergo sterilization processes such as filtration or aseptic processing. The final insulin nanoparticle formulation is then filled into suitable containers, often vials or sterile syringes, under aseptic conditions.

### Microemulsions

The formulation can include microemulsions. Microemulsions are clear, thermodynamically stable mixtures of oil, water, and surfactants. Intranasal microemulsions can be prepared by combining the oil phase (e.g., lipids), water phase, and surfactants, followed by homogenization or high-energy mixing to form a clear and stable microemulsion. Co-surfactants or co-solvents may be included to enhance stability or solubilize lipophilic drugs. The resulting microemulsion can be filled into appropriate containers for intranasal administration.

The formulation of insulin microemulsions begins by selecting suitable components such as oils, surfactants, and co-surfactants. The choice of components depends on their ability to form a clear, isotropic mixture and their compatibility with insulin. Commonly used oils include medium-chain triglycerides (MCT), while surfactants and co-surfactants can include nonionic or mixtures of nonionic and cationic surfactants.

The process typically starts with the selection and mixing of the oil, surfactants, and co-surfactants in appropriate ratios to achieve a clear and stable microemulsion. The mixture is usually prepared using high-shear mixing techniques, such as high-speed homogenization or sonication, to facilitate the formation of the microemulsion.

Insulin, preferably in its soluble form like insulin lispro or insulin aspart, is then incorporated into the microemulsion. This is typically achieved by adding insulin to the oil phase during the formulation process, ensuring its proper dispersion throughout the microemulsion.

To enhance stability and prevent phase separation, co-solvents or co-surfactants may be included. These components can help maintain the thermodynamic stability of the microemulsion and improve the solubilization of insulin.

Once the microemulsion is formed, it may undergo additional processing steps such as filtration or centrifugation to remove any potential aggregates or impurities. The purified insulin microemulsion is then filled into suitable containers, often vials or sterile syringes, under aseptic conditions.

### In Situ Gelling Systems

The formulation can include in situ gelling systems. In situ gelling systems are liquid formulations that undergo gelation upon contact with nasal mucosal fluids. These systems are typically composed of polymers that form a gel network. The preparation involves dissolving or dispersing the polymer(s) in a suitable solvent or vehicle, along with the drug and other excipients. The mixture is then homogenized, sterilized if required, and filled into appropriate containers. Upon administration, the formulation undergoes gelation due to factors such as temperature change, pH adjustment, or exposure to nasal mucosal fluids.

The formulation of insulin in situ gelling systems begins with the selection of suitable polymers and excipients. Biocompatible and biodegradable polymers, such as thermosensitive polymers or mucoadhesive polymers, are commonly used. Poloxamers (Pluronic®) and carbomers are examples of thermosensitive polymers that undergo gelation upon a temperature change, while chitosan is a mucoadhesive polymer that can enhance nasal retention.

To prepare the in-situ gelling system, the selected polymer is dispersed or dissolved in a suitable solvent or water. Insulin, preferably in its soluble form like insulin lispro or insulin aspart, is then added to the polymer solution. The mixture is thoroughly mixed to ensure uniform dispersion of insulin within the polymer matrix.

To induce gelation upon contact with nasal mucosal fluids, certain triggers can be incorporated into the formulation. These triggers can include pH adjustment, ionic strength modulation, or temperature change. For example, pH-sensitive systems can be designed using acids or bases that cause pH changes in the nasal environment, leading to gel formation.

The formulation may include additional excipients such as buffering agents to maintain the desired pH range, or viscosity modifiers to achieve the desired gel consistency. These excipients help optimize the performance of the in situ gelling system.

Once the insulin in situ gelling system is prepared, it can be filled into suitable containers under aseptic conditions. These containers should allow for controlled and accurate administration of the gel-forming liquid.

### ASPECTS OF THE DISCLOSURE

The present disclosure will be better understood upon reading the following numbered aspects, which should not be confused with the claims. In some instance, the aspects below may be combined with one or more additional aspects or with other aspects described elsewhere in the disclosure and accompanying examples. All such variations and combinations are intended to be covered by the instant disclosure.
Aspect 1. A method of treatment of an eye disease in a mammal in a subject in need thereof, the method comprising the step of administering intranasally to the mammal an effective amount of insulin or an insulin analog or derivative. In some aspects, the subject is a mammal. In some aspects, the subject is a human.
Aspect 2. The method according to any one of Aspects 1-38, wherein the eye disease comprises diabetic retinopathy.
Aspect 3. The method according to any one of Aspects 1-38, wherein the method comprises administering insulin.
Aspect 4. The method according to any one of Aspects 1-38, wherein the method comprises administering an insulin analog.
Aspect 5. The method according to any one of Aspects 1-38, wherein the insulin analog is selected from the group consisting of Insulin Lispro, Insulin Aspart, Insulin Glulisine, Insulin Detemir, Insulin Glargine, Insulin Degludec, Insulin Inhalation Powder, Insulin Human, Insulin Regular, Insulin NPH (Neutral Protamine Hagedorn), Insulin Lente, Insulin Ultralente, Insulin PZI (Protamine Zinc Insulin), Insulin Glucose Solution, Insulin Zinc Suspension, Insulin Semilente, Insulin Extended, Insulin Biphasic, Insulin Combinations, and Insulin Tregopil.
Aspect 6. The method according to any one of Aspects 1-38, wherein the eye disease comprises a diabetic eye disease.
Aspect 7. The method according to any one of Aspects 1-38, wherein the diabetic eye disease is selected from the group consisting of diabetic retinopathy, diabetic macular edema, cataracts, glaucoma, and a combination thereof.
Aspect 8. The method according to any one of Aspects 1-38, further comprising monitoring a progression of the eye disease during a course of treatment.
Aspect 9. The method according to any one of Aspects 1-38, wherein monitoring a progression of the eye disease comprises Dilated Fundus Examination, OCT, FA, ICG, high-resolution retinal photography, visual acuity testing, slit-lamp examination, ophthalmoscopy, contrast sensitivity testing, electroretinogram, or a combination thereof.
Aspect 10. The method according to any one of Aspects 1-38, wherein the treatment results in a reduction of the progression of or a reduction in the presence of characteristic retinal changes, such as microaneurysms, hemorrhages, exudates, and abnormal blood vessels.
Aspect 11. The method according to any one of Aspects 1-38, wherein the treatment results in a reduction of the progression of or a reduction in the presence of fluid accumulation, cysts, and other structural changes associated with diabetic retinopathy.
Aspect 12. The method according to any one of Aspects 1-38, wherein the treatment results in a reduction in the progression of or a reduction in the presence of neovascularization, leakage, and areas of ischemia in the retina.
Aspect 13. The method according to any one of Aspects 1-38, wherein the treatment prevents or slows the progression of a flattening of the b wave in the electroretinogram of the mammal.
Aspect 14. The method according to any one of Aspects 1-38, wherein the reduction is relative to the otherwise same mammal undergoing the otherwise same treatment except without receiving the intranasal insulin.
Aspect 15. The method according to any one of Aspects 1-38, wherein the reduction is relative to the otherwise same mammal undergoing the otherwise same treatment except receiving less than the effective amount of intranasal insulin.
Aspect 16. The method according to any one of Aspects 1-38, wherein the mammal has diabetes and is receiving insulin therapy for the diabetes.
Aspect 17. The method according to any one of Aspects 1-38, wherein the administering step comprises administering the insulin or insulin analog or derivative using an apparatus.
Aspect 18. The method according to any one of Aspects 1-38, wherein the apparatus is a pipette.
Aspect 19. The method according to any one of Aspects 1-38, wherein the apparatus is a micropipette.
Aspect 20. The method according to any one of Aspects 1-38, wherein the apparatus is a polyethylene tube attached to a micropipette.
Aspect 21. The method according to any one of Aspects 1-38, wherein the apparatus is a syringe.
Aspect 22. The method according to any one of Aspects 1-38, wherein the apparatus is an intranasal cannula.
Aspect 23. The method according to any one of Aspects 1-38, wherein the apparatus is a cannula attached to a syringe.
Aspect 24. The method according to any one of Aspects 1-38, wherein the apparatus is a modified nasal atomizer.
Aspect 25. The method according to any one of Aspects 1-38, wherein the apparatus is a mucosal atomizer device.
Aspect 26. The method according to any one of Aspects 1-38, wherein the apparatus is a nasal actuator.
Aspect 27. The method according to any one of Aspects 1-38, wherein the apparatus is a face mask.
Aspect 28. The method according to any one of Aspects 1-38, wherein the apparatus is a nebulizer.
Aspect 29. The method according to any one of Aspects 1-38, wherein the apparatus is an inhalers.
Aspect 30. The method according to any one of Aspects 1-38, wherein the insulin or insulin analog or derivative is intranasally administered as a spray.
Aspect 31. The method according to any one of Aspects 1-38, wherein the insulin or insulin analog or derivative is intranasally administered as an aerosol.
Aspect 32. The method according to any one of Aspects 1-38, wherein the insulin or insulin analog or derivative is intranasally administered as drops.
Aspect 33. The method according to any one of Aspects 1-38, wherein the insulin or insulin analog or derivative is intranasally administered as a puff.
Aspect 34. The method according to any one of Aspects 1-38, wherein the insulin or insulin analog or derivative is intranasally administered as an ointment.
Aspect 35. The method according to any one of Aspects 1-38, wherein the insulin or insulin analog or derivative is intranasally administered in an oxygen treatment.
Aspect 36. The method according to any one of Aspects 1-38, wherein the insulin is selected from the group consisting of rapid acting insulin, short acting insulin, intermediate acting insulin, long-acting insulin, ultra-long acting insulin, premixed insulin, and rapid-acting inhaled insulin.
Aspect 37. The method according to any one of Aspects 1-38, wherein the mammal is a diabetic and the method includes preventing a diabetic eye disease in the mammal.
Aspect 38. The method according to any one of Aspects 1-37, comprising administering a pharmaceutical formulation according to any one of Aspects 39-45.
Aspect 39. A pharmaceutical formulation for intranasal delivery in a subject in need thereof, the formulation comprising an effective amount of an insulin, insulin analog, or insulin derivative to treat an eye disease in the mammal, and a pharmaceutically acceptable excipient or carrier. In some aspects, the subject is a mammal. In some aspects, the subject is a human.
Aspect 40. The pharmaceutical formulation according to any one of Aspects 39-45 in the form of a nasal spray.
Aspect 41. The pharmaceutical formulation according to any one of Aspects 39-45 in the form of a nasal gel.
Aspect 42. The pharmaceutical formulation according to any one of Aspects 39-45 in the form of a nasal powder.
Aspect 43. The pharmaceutical formulation according to any one of Aspects 39-45 in the form of nanoparticles.
Aspect 44. The pharmaceutical formulation according to any one of Aspects 39-45 in the form of a microemulsion.
Aspect 45. The pharmaceutical formulation according to any one of Aspects 39-45 in the form of an in-situ gelling system.
Aspect 46. The use of insulin or an insulin analog or insulin derivative in the manufacture of a medicament for the treatment of an eye disease in a subject in need thereof. In some aspects, the subject is a mammal. In some aspects, the subject is a human.
Aspect 47. The use according to any one of Aspects 46-49, wherein the medicament is formulated for intranasal administration.
Aspect 48. The use according to any one of Aspects 46-49, wherein the eye disease is a diabetic eye disease.
Aspect 49. The use according to any one of Aspects 46-49, wherein the diabetic eye disease is selected from the group consisting of diabetic retinopathy, diabetic macular edema, cataracts, glaucoma, and a combination thereof.
Aspect 50. A kit comprising: an insulin, insulin analog, insulin derivative, or a pharmaceutical formulation according to any one of Aspects 39-45; an apparatus for administering the insulin, insulin analog, insulin derivative, or pharmaceutical formulation; and instructions for the intranasal administration of an effective amount of the insulin, insulin analog, insulin derivative, or pharmaceutical formulation to treat or prevent an eye disease in a subject in need thereof. In some aspects, the subject is a mammal. In some aspects, the subject is a human.
Aspect 51. The kit according to any one of Aspects 50-62, wherein the apparatus is a pipette.
Aspect 52. The kit according to any one of Aspects 50-62, wherein the apparatus is a micropipette.
Aspect 53. The kit according to any one of Aspects 50-62, wherein the apparatus is a polyethylene tube attached to a micropipette.
Aspect 54. The kit according to any one of Aspects 50-62, wherein the apparatus is a syringe.
Aspect 55. The kit according to any one of Aspects 50-62, wherein the apparatus is an intranasal cannula.
Aspect 56. The kit according to any one of Aspects 50-62, wherein the apparatus is a cannula attached to a syringe.
Aspect 57. The kit according to any one of Aspects 50-62, wherein the apparatus is a modified nasal atomizer.
Aspect 58. The kit according to any one of Aspects 50-62, wherein the apparatus is a mucosal atomizer device.
Aspect 59. The kit according to any one of Aspects 50-62, wherein the apparatus is a nasal actuator.
Aspect 60. The kit according to any one of Aspects 50-62, wherein the apparatus is a face mask.
Aspect 61. The kit according to any one of Aspects 50-62, wherein the apparatus is a nebulizer.
Aspect 62. The kit according to any one of Aspects 50-62, wherein the apparatus is an inhaler.

It should be emphasized that the above-described aspects of the present disclosure are merely possible examples of implementations, and are set forth only for a clear understanding of the principles of the disclosure. Many variations and modifications may be made to the above-described aspects of the disclosure without departing substantially from the spirit and principles of the disclosure. All such modifications and variations are intended to be included herein within the scope of this disclosure.

### REFERENCES

1. NCHS. National Health Interview Survey. Accessed October 17. 2021. https://www.cdc.gov/nchs/products/databriefs/db344.htm
2. Ip MS, Domalpally A, Hopkins JJ, Wong P, Ehrlich JS. Long-term effects of ranibizumab on diabetic retinopathy severity and progression. Arch Ophthalmol. Sep 2012;130(9):1145-52. doi:10.1001/archophthalmol.2012.1043
3. Gardner TW, Antonetti DA, Barber AJ, LaNoue KF, Levison SW. Diabetic retinopathy: more than meets the eye. Surv Ophthalmol. Dec 2002;47 Suppl 2: S253-62. doi: 10.1016/s0039-6257(02)00387-9
4. Antonetti DA, Barber AJ, Bronson SK, et al. Diabetic retinopathy: seeing beyond glucose-induced microvascular disease. Diabetes. Sep 2006;55(9):2401-11. doi: 10.2337/db05-1635
5. Barber AJ, Gardner TW, Abcouwer SF. The significance of vascular and neural apoptosis to the pathology of diabetic retinopathy. Invest Ophthalmol Vis Sci. Feb 2011;52(2):1156-63. doi: 10.1167/iovs.10-6293
6. Hernandez C, Simo R. Neuroprotection in diabetic retinopathy. Curr Diab Rep. Aug 2012;12(4):329-37. doi:10.1007/s11892-012-0284-5
7. Whitmire W, Al-Gayyar MM, Abdelsaid M, Yousufzai BK, El-Remessy AB. Alteration of growth factors and neuronal death in diabetic retinopathy: what we have learned so far. Mol Vis. Jan 28 2011;17:300-8.
8. Reiter CE, Wu X, Sandirasegarane L, et al. Diabetes reduces basal retinal insulin receptor signaling: reversal with systemic and local insulin. Diabetes. Apr 2006;55(4)1148-56. doi: 10.2337/diabetes.55.04.06.db05-0744
9. Barber AJ, Nakamura M, Wolpert EB, et al. Insulin rescues retinal neurons from apoptosis by a phosphatidylinositol 3-kinase/Akt-mediated mechanism that reduces the activation of caspase-3. J Biol Chem. Aug 31 2001;276(35):32814-21. doi:10.1074/jbc.M104738200
10. Wu X, Reiter CE, Antonetti DA, Kimball SR, Jefferson LS, Gardner TW. Insulin promotes rat retinal neuronal cell survival in a p70S6K-dependent manner. J Biol Chem. Mar 5 2004;279(10):9167-75. doi:10.1074/jbc.M312397200
11. Zeng HY, Green WR, Tso MO. Microglial activation in human diabetic retinopathy. Arch Ophthalmol. Feb 2008;126(2):227-32. doi:10.1001/archophthalmol.2007.65
12. Imai H, Misra GP, Wu L, Janagam DR, Gardner TW, Lowe TL. Subconjunctivally Implanted Hydrogels for Sustained Insulin Release to Reduce Retinal Cell Apoptosis in Diabetic Rats. Invest Ophthalmol Vis Sci. Dec 2015;56(13):7839-46. doi:10.1167/iovs.15-16998
13. Lochhead JJ, Kellohen KL, Ronaldson PT, Davis TP. Distribution of insulin in trigeminal nerve and brain after intranasal administration. Sci Rep. Feb 22 2019;9(1)2621. doi:10.1038/s41598-019-39191-5
14. Thorne RG, Pronk GJ, Padmanabhan V, Frey WH, 2nd. Delivery of insulin-like growth factor-I to the rat brain and spinal cord along olfactory and trigeminal pathways following intranasal administration. Neuroscience. 2004;127(2):481-96. doi:10.1016/j.neuroscience.2004.05.029
15. Porta M, Bandello F. Diabetic retinopathy: a clinical update. Diabetologia. 2002;45:1617-23
16. CDC. National Diabetes Statistics Report 2020: Estimates of Diabetes and Its Burden in the United States. Accessed Oct. 16, 2021. https://www.cdc.gov/diabetes/pdfs/data/statistics/national-diabetes-statistics-report.pdf
17. Boyle JP, Thompson TJ, Gregg EW, Barker LE, Williamson DF. Projection of the year 2050 burden of diabetes in the US adult population: dynamic modeling of incidence, mortality, and prediabetes prevalence. Popul Health Metr. Oct 22 2010;8:29. doi:10.1186/1478-7954-8-29
18. Luo H, Bell RA, Garg S, Cummings DM, Patil SP, Jones K. Trends and Racial/Ethnic Disparities in Diabetic Retinopathy Among Adults with Diagnosed Diabetes in North Carolina, 2000-2015. N C Med J. 2019 Mar-Apr 2019;80(2):76-82. doi:10.18043/ncm.80.2.76
19. CDC. Diabetes State Burden Toolkit: Technical Report. Accessed October 17, 2021. https://nccd.cdc.gov/Toolkit/DiabetesBurden/images/docs/technical_documentation.pdf
20. Yau JW, Rogers SL, Kawasaki R, et al. Global prevalence and major risk factors of diabetic retinopathy. Diabetes Care. Mar 2012;35(3):556-64. doi:10.2337/dc11-1909
21. Carter JS, Pugh JA, Monterrosa A. Non-insulin-dependent diabetes mellitus in minorities in the United States. Ann Intern Med. Aug 1 1996;125(3):221-32. doi:10.7326/0003-4819-125-3-199608010-00011
22. Stitt AW, Curtis TM, Chen M, et al. The progress in understanding and treatment of diabetic retinopathy. Prog Retin Eye Res. Mar 2016;51:156-86. doi: 10.1016/j.preteyeres.2015.08.001
23. Duh EJ, Sun JK, Stitt AW. Diabetic retinopathy: current understanding, mechanisms, and treatment strategies. JCI Insight. Jul 20 2017;2(14)doi:10.1172/jci.insight.93751
24. Gardiner TA, Stitt AW, Anderson HR, Archer DB. Selective loss of vascular smooth muscle cells in the retinal microcirculation of diabetic dogs. Br J Ophthalmol. Jan 1994;78(1):54-60. doi:10.1136/bjo.78.1.54
25 Mizutani M, Kern TS, Lorenzi M. Accelerated death of retinal microvascular cells in human and experimental diabetic retinopathy. J Clin Invest. Jun 15 1996;97(12):2883-90. doi:10.1172/JCI118746
26. Han Y, Adams AJ, Bearse MA, Schneck ME. Multifocal electroretinogram and short-wavelength automated perimetry measures in diabetic eyes with little or no retinopathy. Arch Ophthalmol. Dec 2004;122(12):1809-15. doi:10.1001/archopht.122.12.1809
27. Harrison WW, Bearse MA, Ng JS, et al. Multifocal electroretinograms predict onset of diabetic retinopathy in adult patients with diabetes. Invest Ophthalmol Vis Sci. Feb 2011;52(2) 772-7. doi:10.1167/iovs.10-5931
28. Lim SW, Cheung N. Birth weight and retinal vascular changes. Hypertension. Jun 2008;51(6):e56; author reply e57. doi: 10.1161/HYPERTENSIONAHA. 108.112839
29. Reiter CE, Sandirasegarane L, Wolpert EB, et al. Characterization of insulin signaling in rat retina in vivo and ex vivo. Am J Physiol Endocrinol Metab. Oct 2003;285(4):E763-74. doi:10.1152/ajpendo.00507.2002
30. Diabetes C, Complications Trial Research G, Nathan DM, et al. The effect of intensive treatment of diabetes on the development and progression of long-term complications in insulin-dependent diabetes mellitus. N Engl J Med. Sep 30 1993;329(14):977-86. doi:10.1056/NEJM199309303291401
31. Zhang L, Krzentowski G, Albert A, Lefebvre PJ. Risk of developing retinopathy in Diabetes Control and Complications Trial type 1 diabetic patients with good or poor metabolic control. Diabetes Care. Jul 2001;24(7):1275-9. doi:10.2337/diacare.24.7.1275
32. Simon AC, DeVries JH. The future of basal insulin supplementation. Diabetes Technol Ther. Jun 2011;13 Suppl 1:S103-8. doi:10.1089/dia.2010.0251
33. Cryer PE. Hypoglycemia is the limiting factor in the management of diabetes. Diabetes Metab Rev. 1999;15:42-6.
34. Koevary SB. Nussey J, Lake S. Accumulation of topically applied porcine insulin in the retina and optic nerve in normal and diabetic rats. Invest Ophthalmol Vis Sci. Mar 2002;43(3):797-804.
35. Urtti A. Challenges and obstacles of ocular pharmacokinetics and drug delivery. Adv Drug Deliv Rev. November 15 2006;58(11):1131-5. doi:10.1016/j.addr.2006.07.027
36. Prasad AG, Schadlu R, Apte RS. Intravitreal pharmacotherapy: applications in retinal disease. Compr Ophthalmol Update. Sep-Oct 2007;8(5):259-69.
37. Fan LW, Carter K, Bhatt A, Pang Y. Rapid transport of insulin to the brain following intranasal administration in rats. Neural Regen Res. Jun 2019;14(6):1046-1051. doi:10.4103/1673-5374.250624
38. Khan RS, Dine K, Bauman B, et al. Intranasal Delivery of A Novel Amnion Cell Secretome Prevents Neuronal Damage and Preserves Function In A Mouse Multiple Sclerosis Model. Sci Rep. Jan 31 2017;7:41768. doi:10.1038/srep41768
39. Grinblat GA, Khan RS, Dine K, Wessel H, Brown L, Shindler KS. RGC Neuroprotection Following Optic Nerve Trauma Mediated By Intranasal Delivery of Amnion Cell Secretome. Invest Ophthalmol Vis Sci. May 1 2018;59(6):2470-2477. doi:10.1167/iovs.18-24096
40. Bogdanov P, Corraliza L, Villena JA, et al. The db/db mouse: a useful model for the study diabetic of retinal neurodegeneration. PLoS One. 2014;9(5):e97302. doi:10.1371/journal.pone.0097302
41. Phipps JA, Fletcher EL, Vingrys AJ. Paired-flash identification of rod and cone dysfunction in the diabetic rat. Invest Ophthalmol Vis Sci. December 2004;45(12) 4592-600. doi:10.1167/iovs.04-0842
42. Touitou E, Illum L. Nasal drug delivery. Drug Deliv Transl Res. 2013 Feb;3(1):1-3. doi: 10.1007/s13346-012-0111-1. PMID:25787862.
43. S. Khan, K. Patil, P. Yeole, R. Gaikwad. Brain targeting studies on buspirone hydrochloride after intranasal administration of mucoadhesive formulation in rats. J. Pharm. Pharmacol., 61 (2009), pp. 669-675, 10.1211/jpp/61.05.0017

### EXAMPLES

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to the examples. On the contrary, the intent is to cover all alternatives, modifications, and equivalents included within the spirit and scope of the present disclosure and examples.

### Methods

Female Sprague Dawley rats were deprived of food for 12-18 hours and anesthetized with IP ketamine/xylazine. Rats (n=4) were placed in the supine position on a heating pad (37 degrees Celsius) and one 20 μl drop of FITC-insulin (total dose 6 Units/40 μl saline) was pipetted into each naris 5 minutes apart. The control group (n=4) received equivalent doses of saline. One hour after the first drop was intranasally given, rats were sacrificed, enucleated, and the brain was removed. The presence of FITC insulin was examined in cryosections of brain and whole eye, using confocal microscopy. Deposition of FITC-insulin in neural retina and choroid was further characterized by immunohistochemical co-localization studies.

Female Sprague Dawley rats that were fed ad-libitum were anesthetized with an intraperitoneal injection of ketamine/xylazine prior to intranasal insulin administration. Rats were sacrificed by trans-cardial perfusion and the eyes and brain were harvested for histological assessment one hour after FITC insulin administration.

13-week old control C57/BL6 and diabetic C57BL/KsJ-db/db mice were treated with intranasal saline or 2U intranasal insulin daily for 10 weeks. ERG was taken before and at the end of intranasal saline or insulin treatment.

To examine whether intranasal insulin delivery could protect against retinal degeneration in diabetic mice, BKS.Cg-Dock7^{m}+/+Lepr^{db}/J diabetic mice were given intranasal insulin once daily for 10 weeks beginning at 13 weeks of age. Male C57B6 mice administered intranasal saline once daily were taken as a negative control. Thirty male BKS.Cg-Dock7^{m}+/+Lepr^{db}/J diabetic mice were divided into three groups of n=10 and, beginning at 13 weeks of age, were administered daily for 10 weeks intranasal saline (positive control group), 1U Insulin (low dose group), or 2U Insulin (high dose group). Blood glucose measurements were taken for all groups before and 30 minutes after intranasal administration of either the saline or insulin. Dark adapted electroretinogram (ERG) were taken for all groups at 11 weeks and at 22 weeks of age. All mice were sacrificed at 23 weeks of age. One eye from each mouse was submersion fixed for TUNEL and IHC analysis, and the other eye was frozen for RNA sequencing analysis.

12 male 6-week old C57BL/6 mice will be given intranasal insulin manually without anesthesia by holding the pup in a supine position and placing a 10 μl drop of the solution to cover the opening of both nostrils, and not forcibly into the nares. The mouse is held supine for 5 seconds, allowing the mouse to inhale a volume suitable for their size. They are allowed to recover for 5 mins before repeating the procedure. There will be four mice in each experimental group. In each group, four mice will be given one 10 μl drop of 1) FITC insulin (total dose 2 Units/20 μl saline), 2) FITC insulin (total dose 1 Unit/20 μl saline), and 3) 10 μl saline (control) pipetted into each naris 5 min apart. 30 minutes after the first drop is intranasally given, mice will be sacrificed by trans-cardial perfusion and the eyes and brain harvested for histological assessment. The presence of FITC insulin will be detected in cryosections of brain and whole eye, using direct imaging, immunohistochemistry, Western blot, and confocal microscopy. Brain sections will serve as positive controls.

### Results

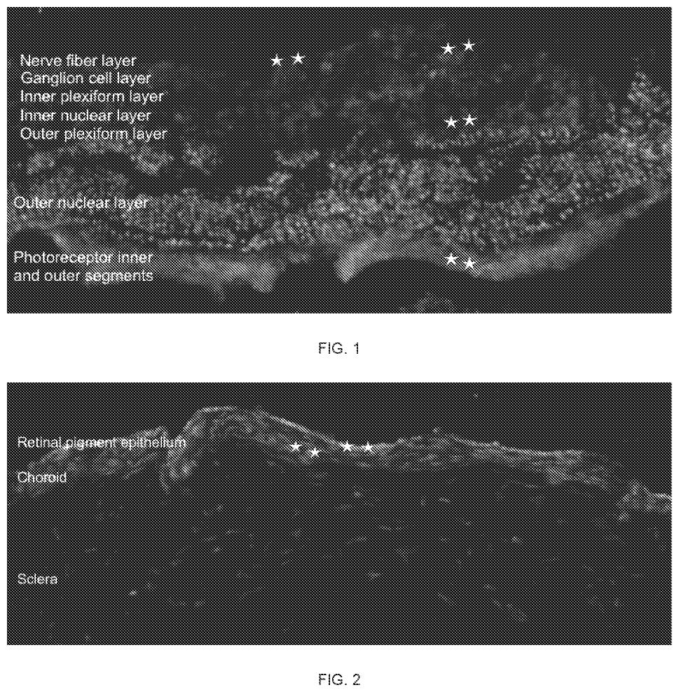

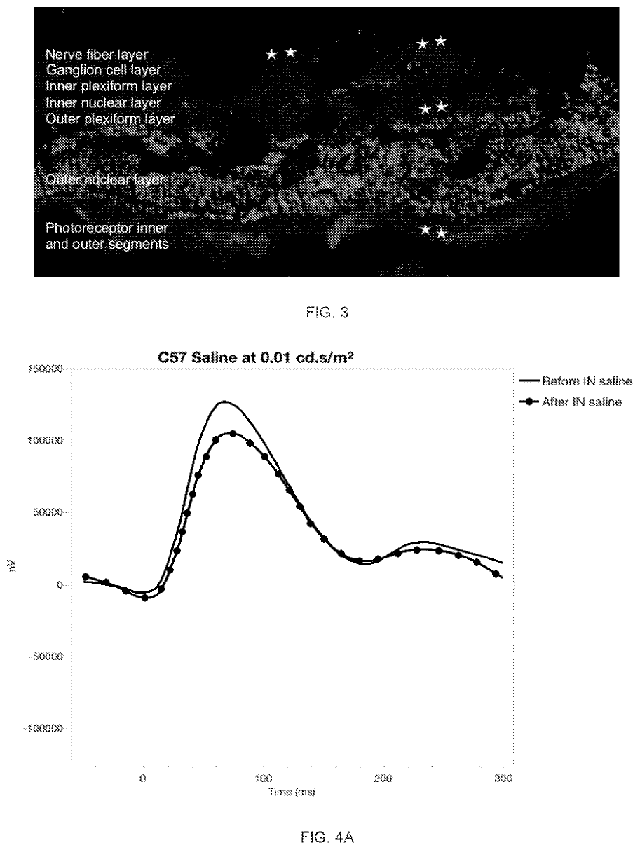

Sixty minutes after intranasal administration of FITC-insulin, fluorescent signal was evident in both ocular and brain tissue. Ocular and brain tissue from the control group was negative for FITC signal. As shown in FIG. 1 and FIG. 2, FITC patterning indicated deposition of insulin predominantly in the retinal pigment epithelium and near outer segments of rods and cones. Lower intensity deposition was observed in the choriocapillaris, the inner and outer plexiform layers, and the nerve fiber layer. Brain sections, which served as positive controls, confirmed the deposition of intranasal insulin in the hippocampus, cortex, and hypothalamus.

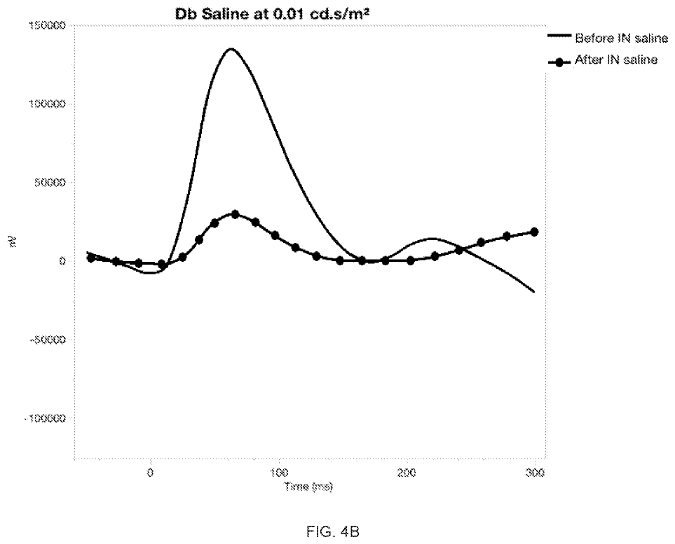

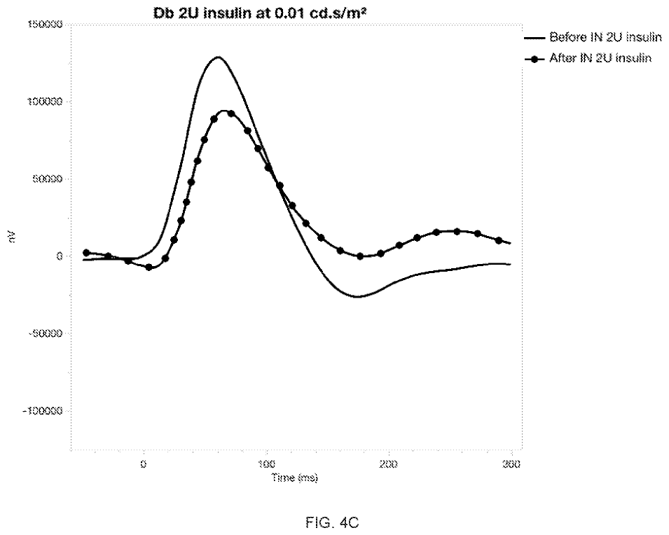

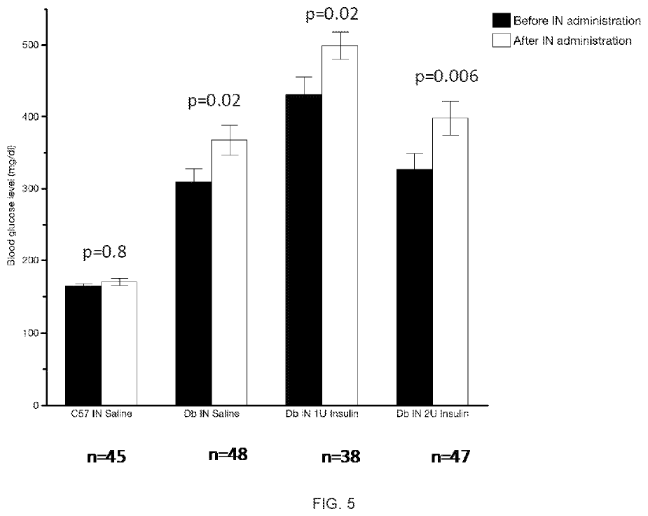

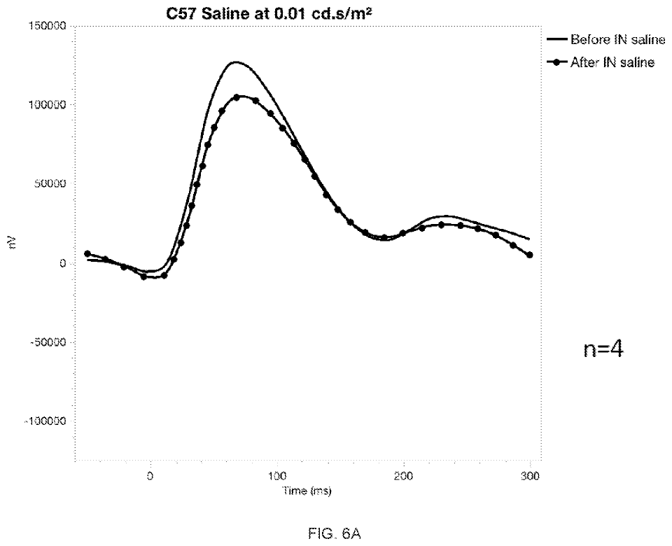

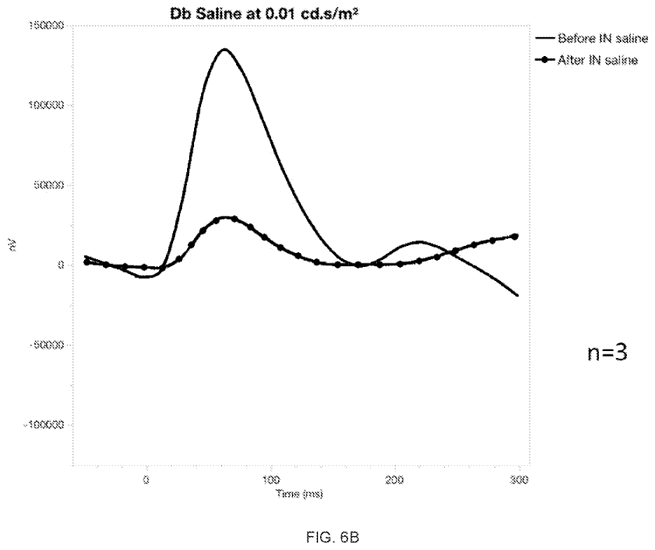

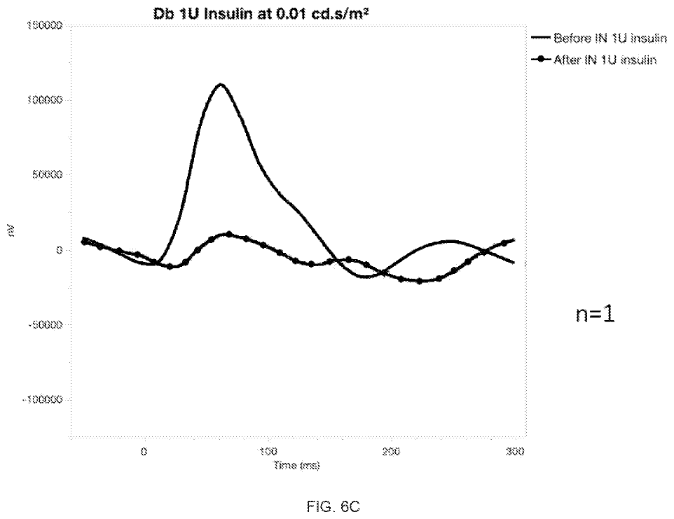

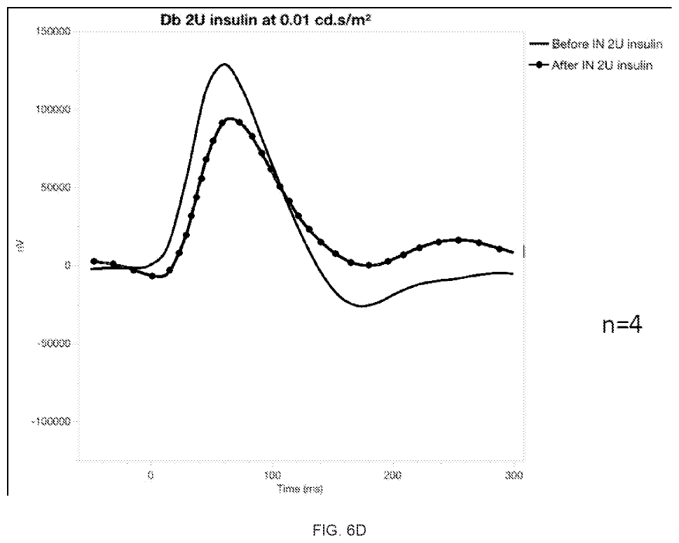

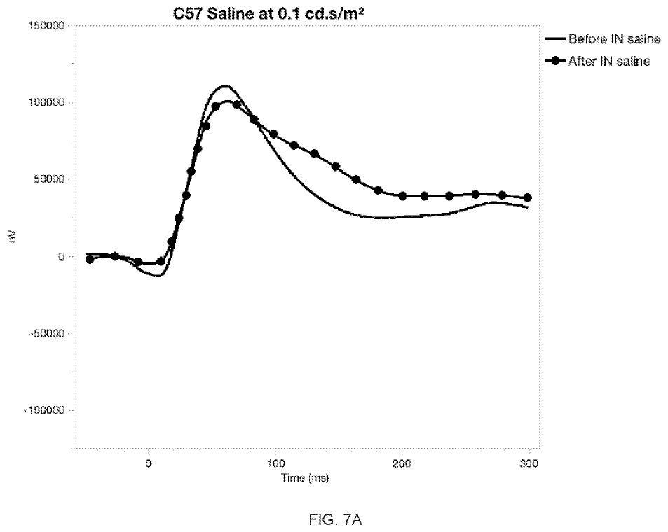

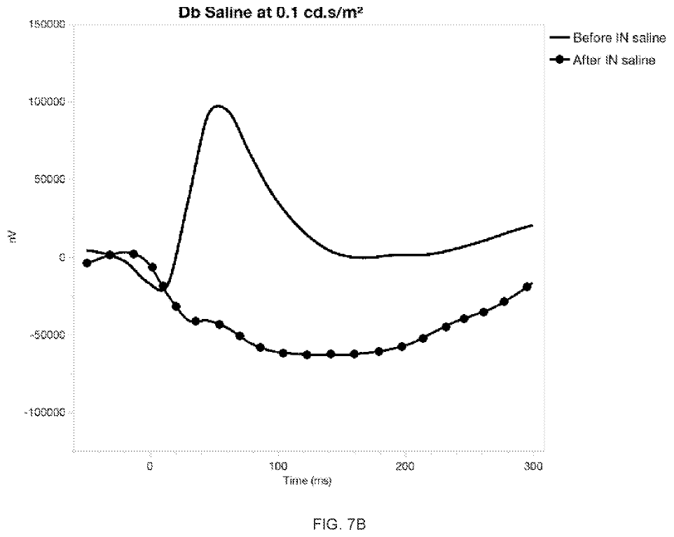

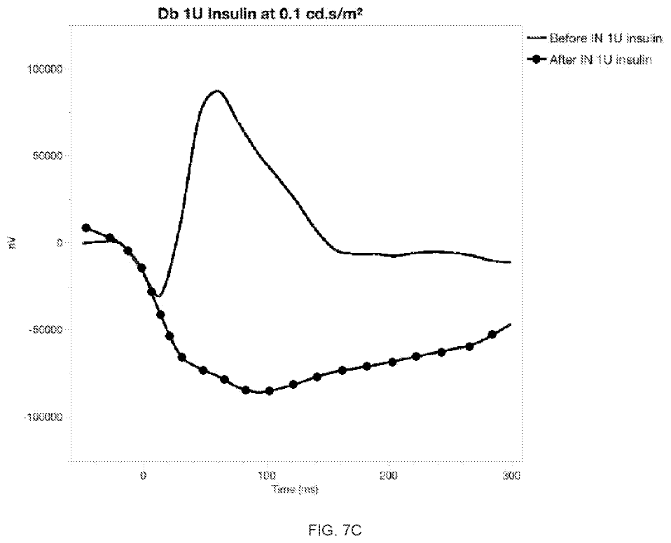

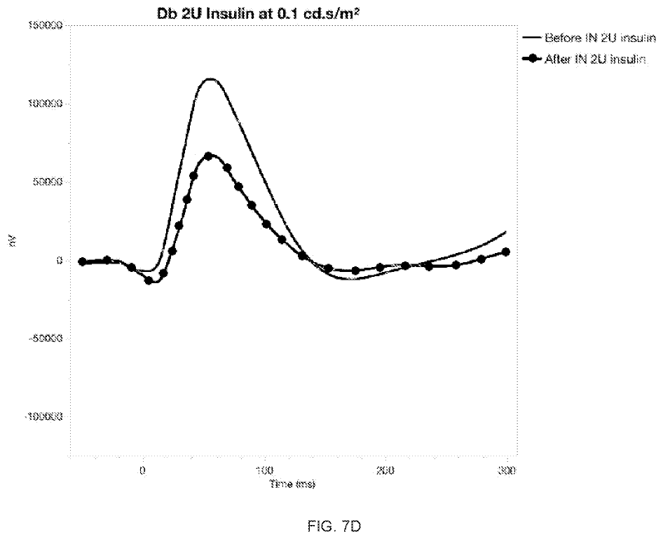

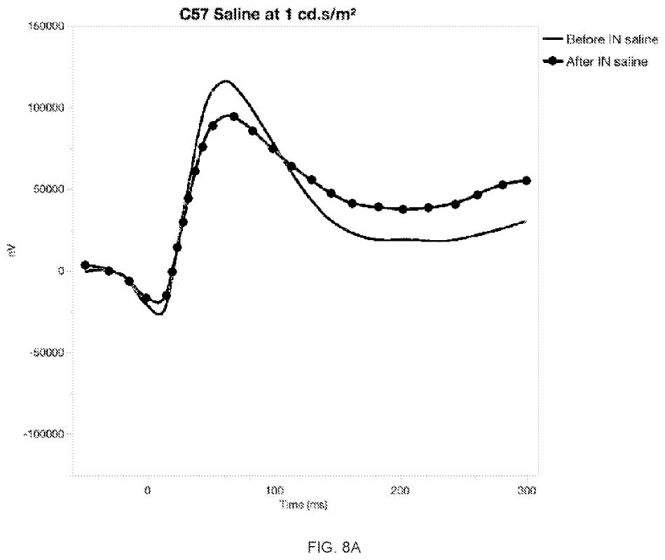

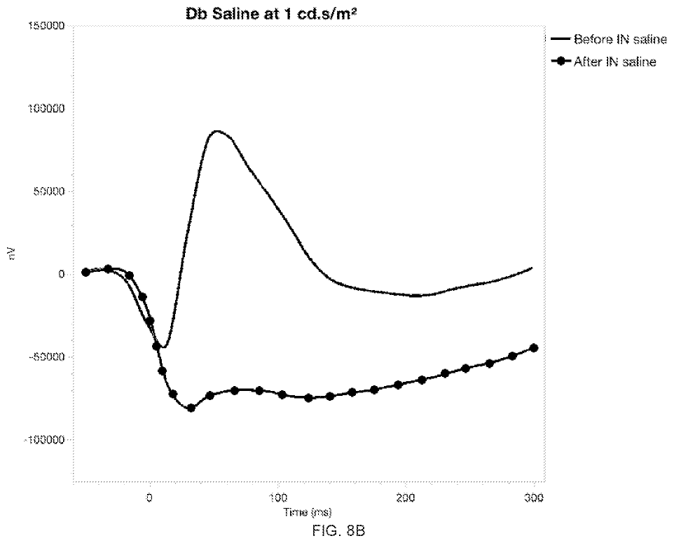

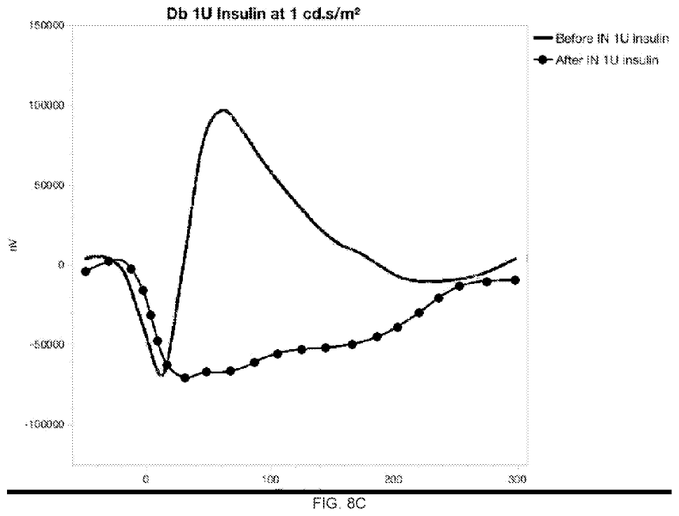

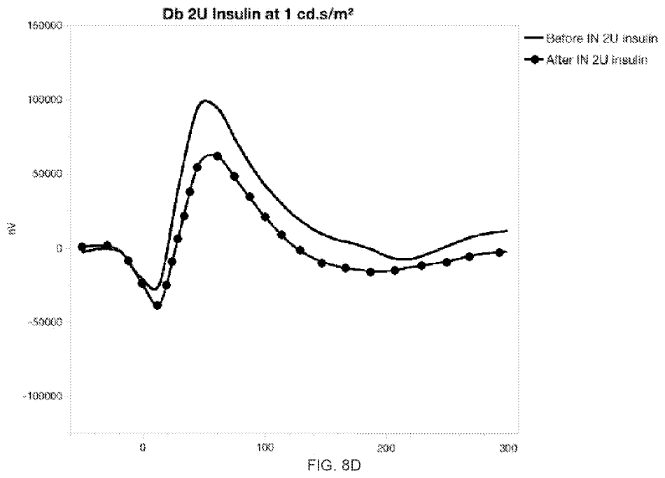

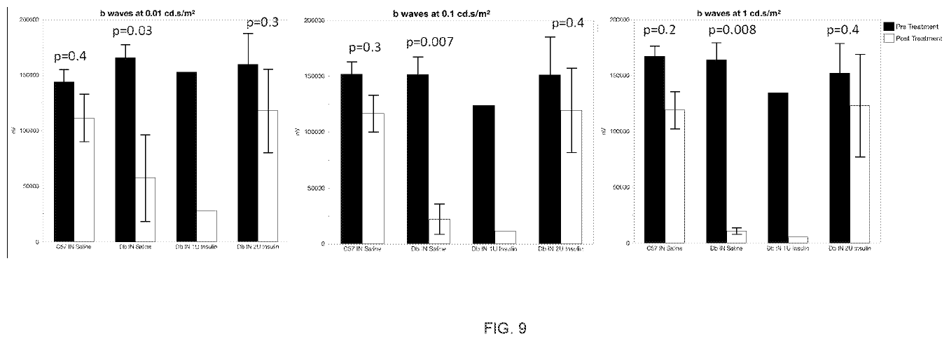

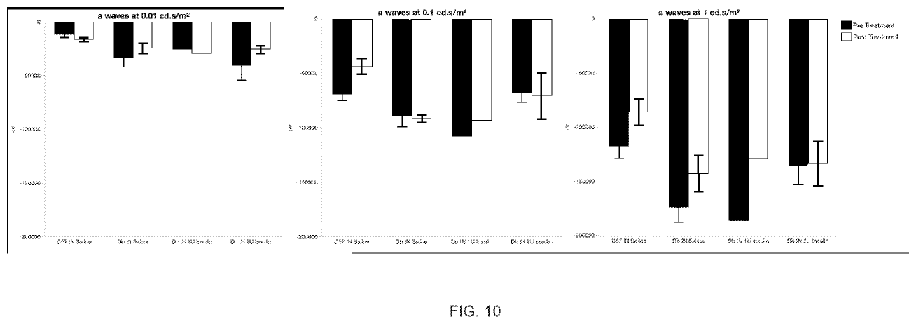

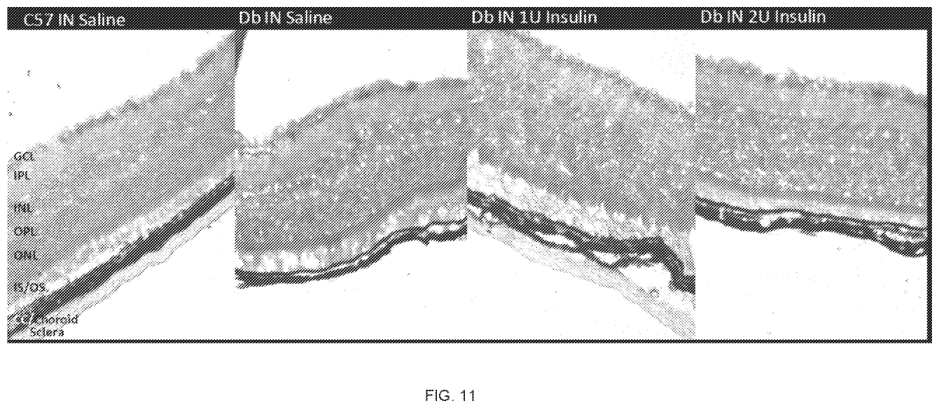

FIGS. 4A-4C demonstrate the electroretinogram results from 13-week old control C57/BL6 and diabetic mice that were treated with intranasal saline or 2U intranasal insulin daily for 10 weeks. ERG was taken before and at the end of intranasal saline or insulin treatment. In the negative control C57/BL6 (n=4) group treated with intranasal saline (FIG. 4A), there was no change in a or b waves after treatment (comparison of b waves, p=0.4). In the positive control diabetic C57BL/KsJ-db/db (n=3) group treated with intranasal saline (FIG. 4B), there was no change in a wave but a precipitous drop in b wave (comparison of b waves, p=0.03) after treatment. In the diabetic C57BL/KsJ-db/db (n=4), there was no change in a or b waves (comparison of b waves, p=0.3) after treatment (FIG. 4C). Daily 2U intranasal insulin appears to prevent the flattening of b waves in the diabetic group. B-wave on the ERG is a result of light evoked depolarization of the bipolar cells in the inner retina.

### Conclusions

The disclosed invention teaches that intranasal insulin can be rapidly delivered to the retina and choriocapillaris within one hour of administration and serve as a treatment for retinal diseases. These data suggest that intranasal delivery is an efficient method for early or supplemental insulin therapy in diabetic retinopathy.

### Proof of Concept

12 male 6-week old C57BL/6 mice will be given intranasal insulin manually without anesthesia by holding the pup in a supine position and placing a 10 μl drop of the solution to cover the opening of both nostrils, and not forcibly into the nares. The mouse is held supine for 5 seconds, allowing the mouse to inhale a volume suitable for their size. They are allowed to recover for 5 mins before repeating the procedure. There will be four mice in each experimental group. In each group, four mice will be given one 10 μl drop of 1) FITC insulin (total dose 2 Units/20 μl saline), 2) FITC insulin (total dose 1 Unit/20 μl saline), and 3) 10 μl saline (control) pipetted into each naris 5 min apart. 30 minutes after the first drop is intranasally given, mice will be sacrificed by trans-cardial perfusion and the eyes and brain harvested for histological assessment. The presence of FITC insulin will be detected in cryosections of brain and whole eye, using direct imaging, immunohistochemistry, Western blot, and confocal microscopy. Brain sections will serve as positive controls.

1. A method of treatment of an eye disease in a mammal in need thereof, the method comprising the step of administering intranasally to the mammal an effective amount of insulin or an insulin analog or derivative.

2. The method according to claim 1, wherein the eye disease comprises diabetic retinopathy.

3. The method according to claim 1, wherein the method comprises administering insulin.

4. The method according to claim 1, wherein the method comprises administering an insulin analog.

5. The method according to claim 4, wherein the insulin analog is selected from the group consisting of Insulin Lispro, Insulin Aspart, Insulin Glulisine, Insulin Detemir, Insulin Glargine, Insulin Degludec, Insulin Inhalation Powder, Insulin Human, Insulin Regular, Insulin NPH (Neutral Protamine Hagedorn), Insulin Lente, Insulin Ultralente, Insulin PZI (Protamine Zinc Insulin), Insulin Glucose Solution, Insulin Zinc Suspension, Insulin Semilente, Insulin Extended, Insulin Biphasic, Insulin Combinations, and Insulin Tregopil.

6. The method according to claim 1, wherein the eye disease comprises a diabetic eye disease.

7. The method according to claim 6, wherein the diabetic eye disease is selected from the group consisting of diabetic retinopathy, diabetic macular edema, cataracts, glaucoma, and a combination thereof.

8. (canceled)

9. (canceled)

10. (canceled)

11. The method according to claim 1, wherein the treatment results in a reduction of the progression of or a reduction in the presence of fluid accumulation, cysts, and other structural changes associated with diabetic retinopathy.

12. The method according to any one of claims claim 1, wherein the treatment results in a reduction in the progression of or a reduction in the presence of neovascularization, leakage, and areas of ischemia in the retina.

13. The method according to claim 1, wherein the treatment prevents or slows the progression of a flattening of the b wave in the electroretinogram of the mammal.

14. The method according to claim 13 wherein the reduction is relative to the otherwise same mammal undergoing the otherwise same treatment except without receiving the intranasal insulin.

15. The method according to claim 13 wherein the reduction is relative to the otherwise same mammal undergoing the otherwise same treatment except receiving less than the effective amount of intranasal insulin.

16. The method according to claim 1, wherein the mammal has diabetes and is receiving insulin therapy for the diabetes.

17. The method according to claim 1, wherein the administering step comprises administering the insulin or insulin analog or derivative using an apparatus selected from the group consisting of a pipette, a micropipette, a syringe, an intranasal cannula, a nasal atomizer, a nasal actuator, a face mask, a nebulizer, an inhaler, and a combination thereof.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The method according to claim 1, wherein the insulin or insulin analog or derivative is intranasally administered as a spray, aerosol, drop, puff, ointment, or oxygen treatment.

31. (canceled)

32. (canceled)

33. (canceled)

34. (canceled)

35. (canceled)

36. (canceled)

37. The method according to claim 1, wherein the mammal is a diabetic and the method includes preventing a diabetic eye disease in the mammal.

38. (canceled)

39. A pharmaceutical formulation for intranasal delivery in a mammal in need thereof, the formulation comprising
a. an effective amount of an insulin, insulin analog, or insulin derivative to treat an eye disease in the mammal, and
b. a pharmaceutically acceptable excipient.

40. (canceled)

41. (canceled)

42. (canceled)

43. (canceled)

44. (canceled)

45. (canceled)

46. (canceled)

47. (canceled)

48. (canceled)

49. (canceled)

50. A kit comprising:
a. an insulin, insulin analog, insulin derivative, or a pharmaceutical formulation according to claim 1;
b. an apparatus for administering the insulin, insulin analog, insulin derivative, or pharmaceutical formulation; and
c. instructions for the intranasal administration of an effective amount of the insulin, insulin analog, insulin derivative, or pharmaceutical formulation to treat or prevent an eye disease in a mammal.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
